(19) United States
(12) Patent Application Publication
(10) Pub. No.: US 2026/0354976 A1
(43) Pub. Date: Nov. 12, 2026

(54) DELIVERY ASSEMBLIES WITH ULTRASOUND SENSORS

(71) Applicant: Edwards Lifesciences Corporation, Irvine, CA (US)

(72) Inventors: David Maimon, Atlit (IL); Elazar Levi Schwarcz, Netanya (IL); Ilan Leshecz, Hadera (IL); Halit Yaakobovich, Netanya (IL); Noa Axelrod Manela, Kadima (IL)

(73) Assignee: Edwards Lifesciences Corporation, Irvine, CA (US)

(21) Appl. No.: 19/724,485

(22) Filed: Jun. 29, 2026

Related U.S. Application Data
(63) Continuation of application No. PCT/US2025/011182, filed on Jan. 10, 2025.
(60) Provisional application No. 63/619,894, filed on Jan. 11, 2024.

(51) Int. Cl. A61F 2/24; A61B 5/00; A61B 5/01; A61B 8/00; A61B 8/08; A61B 8/12

(52) U.S. Cl. CPC A61F 2/2496; A61B 5/01; A61B 5/6853; A61B 8/0883; A61B 8/12; A61B 8/445; A61F 2/2433

(57) ABSTRACT

The present disclosure relates to delivery assemblies that include ultrasound sensors. In an example, the delivery assembly comprises a balloon catheter, an inflatable balloon mounted on the balloon catheter, and at least one ultrasound sensor configured to move between a first position which is axially offset from an intermediate section of the balloon, and a second position which is axially aligned with the intermediate section inside a cavity of the balloon.

### CROSS-REFERENCE TO RELATED APPLICATIONS

This application is a continuation of International Application No. PCT/US 2025/011182, filed Jan. 10, 2025, which claims the benefit of U.S. Provisional Application No. 63/619,894, filed Jan. 11, 2024, which is incorporated by reference herein.

### FIELD

The present disclosure relates to apparatuses and methods that can be used in the treatment of heart valve disease, including balloon valvuloplasty and the delivery of transcatheter heart valves.

### BACKGROUND

Heart valve disease is a serious problem that involves the malfunction of one or more valves of the heart. The malfunction can manifest itself in a variety of manners. For example, valve stenosis is the calcification or narrowing of a native heart valve. As a result, the native heart valve is not able to completely open and blood flow through the native valve is impeded or restricted. Another example of heart valve disease is valve insufficiency. Valve insufficiency is the failure of a native heart valve to close properly to prevent leaking, or backflow, of blood through the valve.

Various methods have been developed to treat heart valve disease. Some of these methods require a balloon member that is expanded within the native heart valve. For example, a balloon member can be used in a valvuloplasty procedure where the balloon member is positioned within the native heart valve and expanded to increase the opening size (i.e., flow area) of the native heart valve and thereby improve blood flow. Another procedure that can be performed is a valve replacement, in which a native heart valve is replaced by an artificial heart valve. The implantation of an artificial heart valve in the heart can also involve the expansion of a balloon member in the valve annulus. For example, the balloon member can be used to increase the size of the native valve prior to implantation of the artificial valve and/or it can be used to expand and deploy the artificial valve itself.

### SUMMARY

When implanting a prosthetic valve, such as a balloon expandable valve, it is desirable to expand the valve to a maximum size allowed by the patient's anatomical considerations, in order to avoid paravalvular leakage or other unfavorable hemodynamic phenomena across the valve that may be associated with a mismatch between the valve's expansion diameter and the surrounding tissue, while mitigating the risk of annular rupture that may result from over-expansion.

One potential technique for mitigating the risk of mismatch between a prosthetic valve's expansion diameter and the surrounding tissue, such as the diameter of a native annulus, involves measuring, in real-time, the balloon's or valve's expansion diameter, by an ultrasound sensor placed inside the balloon's cavity However, adding a components such as the ultrasound sensor, inside the balloon, over which the prosthetic valve is crimped, may negatively affect the overall crimped profile, making it harder to pass the delivery assembly through narrow portions of the patient's vasculature.

In some examples, a delivery assembly is provided, the delivery assembly comprising a delivery apparatus. In some examples, the delivery apparatus comprises: a balloon catheter defining a balloon catheter lumen; an inflatable balloon mounted on the balloon catheter and defining a balloon cavity that is in fluid communication with the balloon catheter lumen.

In some examples, the balloon comprises: a balloon proximal section attached to the balloon catheter; a balloon intermediate section extending distally from the balloon proximal section; and a balloon distal section extending distally from the balloon intermediate section.

In some examples, the delivery apparatus comprises at least one ultrasound sensor configured to move between a first position which is axially offset from the balloon intermediate section, and a second position which is axially aligned with the balloon intermediate section inside the balloon cavity.

In some examples, the balloon is configured to transition between a deflated state and an inflated state, wherein the ultrasound sensor is configured to be in the first position in the deflated state of the balloon, and to be in the second position in the inflated state of the balloon.

In some examples, the delivery apparatus further comprises a sleeve extending proximally from the at least one sensor.

In some examples, the delivery apparatus further comprises a stopper disposed around the nosecone shaft inside the balloon cavity, wherein the stopper is axially positioned between the balloon proximal section and the balloon distal section.

In some examples, the at least one ultrasound sensor comprises at least two ultrasound transducers which are diametrically opposite to each other.

In some examples, a method is provided, the method comprising: advancing a delivery assembly that comprises a delivery apparatus comprising a balloon mounted on a balloon catheter and at least one ultrasound sensor, to a target site of treatment, while the at least one ultrasound sensor is in a first position which is axially offset from an intermediate balloon section of the balloon; and inflating the balloon by streaming inflation fluid into a cavity of the balloon. In some examples, the method comprises axially moving the at least one ultrasound sensor to a second position which is aligned with the intermediate balloon section inside the balloon cavity.

In some examples, the advancing the delivery assembly comprises maintaining a spring attached to the at least one ultrasound sensor in a compressed loaded state configured to distally bias the at least one ultrasound sensor towards the second position.

In some examples, the axially moving the at least one ultrasound sensor comprises injecting the inflation fluid into a sleeve extending proximally from the at least one ultrasound sensor, such that the inflation fluid impinges against a proximal surface of the at least one ultrasound sensor and biases the at least one ultrasound sensor distally towards the second position.

In some examples, the at least one ultrasound sensor further comprises a proximally tapering sensor proximal portion, and wherein the advancing the delivery assembly further comprises wedging the sensor proximal portion against the inflow end of the prosthetic valve.

In some examples, a delivery assembly is provided, the delivery assembly comprising a delivery apparatus. In some examples, the delivery apparatus comprises: a balloon catheter defining a balloon catheter lumen; an inflatable balloon mounted on the balloon catheter and defining a balloon cavity that is in fluid communication with the balloon catheter lumen; and at least one ultrasound sensor. In some examples, the delivery apparatus further comprises a temperature sensor.

In some examples, the sensor data unit is configured, based at least in part on an output of the at least one ultrasound sensor, to determine a diameter indication of the balloon.

In some examples, the determination of the diameter indication of the balloon is further based on the output of the temperature sensor.

In some examples, the delivery apparatus further comprises a flow sensor, the determination of the diameter indication of the balloon further based on the output of the flow sensor.

In some examples, a method is provided, the method comprising: advancing a delivery assembly that comprises a delivery apparatus comprising a balloon mounted on a balloon catheter and at least one ultrasound sensor, to a target site of treatment; inflating the balloon by streaming inflation fluid into a cavity of the balloon; and measuring a temperature of the inflation fluid.

In some examples, the method comprises, based at least in part on an output of the at least one ultrasound sensor and the measured temperature of the inflation fluid, determining a diameter indication.

In some examples, the determined diameter indication is of one or more of: the balloon; an anatomical wall around the balloon; or a prosthetic valve disposed around the balloon.

In some examples, the method further comprises: measuring a flow velocity of the inflation fluid; and adjusting the measured temperature based at least in part on the measured flow velocity, the determination of the diameter indication based at least in part on the adjusted temperature.

In some examples, a delivery assembly is provided, the delivery assembly comprises a delivery apparatus. In some examples, the delivery apparatus comprises: a balloon catheter defining a balloon catheter lumen; an inflatable balloon mounted on the balloon catheter and defining a balloon cavity that is in fluid communication with the balloon catheter lumen; and at least one first ultrasound transducer.

In some examples, the delivery apparatus comprises at least one second ultrasound transducer axially offset from the at least one first ultrasound transducer.

In some examples, a sensor data unit is configured to: control the at least one first ultrasound transducer to generate ultrasound waves; and detect the ultrasound waves at the at least one second ultrasound transducer.

In some examples, the sensor data unit is configured, based at least in part on the detected ultrasound waves, to determine a diameter indication of an anatomical wall around the balloon.

In some examples, the delivery assembly comprises a refractive element comprising a refractive surface positioned between the at least one first ultrasound transducer and the at least one second ultrasound transducer such that a line of sight between the at least one first ultrasound transducer and the at least one second ultrasound transducer is blocked.

In some examples, a method is provided, the method comprising: advancing a delivery assembly to a target site of treatment, wherein the delivery assembly comprises a delivery apparatus comprising a balloon mounted on a balloon catheter, at least one first ultrasound transducer and at least one second ultrasound transducer axially offset from the at least one first ultrasound transducer.

In some examples, the method further comprises: inflating the balloon by streaming inflation fluid into a cavity of the balloon; and measuring a temperature of the inflation fluid.

In some examples, the method further comprises: generating ultrasound waves using the at least one first ultrasound transducer; and detecting the ultrasound waves using the at least one second ultrasound transducer.

In some examples, the method further comprises, based at least in part on the detected ultrasound waves, determining a diameter indication of the balloon.

In some examples, the method further comprises, based at least in part on the detected ultrasound waves, determining a diameter indication of an anatomical wall around the balloon.

In some examples, the method further comprises, based at least in part on the detected ultrasound waves, determining a diameter indication of a prosthetic valve disposed around the balloon.

In some examples, a method or device can include any of the features recited in Examples 1-148 below.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A is a perspective view of an exemplary prosthetic valve.

FIG. 1B is a perspective view of a frame of the prosthetic valve of FIG. 1A.

FIG. 2A shows an exemplary delivery assembly comprising a delivery apparatus carrying a prosthetic valve.

FIG. 2B shows the exemplary delivery assembly of FIG. 2A further comprising a fluid passageway coupled to a fluid source.

FIG. 3A is a high-level perspective view of an exemplary delivery apparatus equipped with an ultrasound sensor.

FIG. 3B is a high-level perspective cut-away view of a portion of the delivery apparatus of FIG. 3A.

FIG. 4A is a perspective cut-away view of a distal portion of a delivery apparatus with an exemplary ultrasound sensor disposed inside a balloon thereof.

FIG. 4B is an enlarged perspective view of the ultrasound sensor of FIG. 4A.

FIG. 4C is a cross sectional view across line 4C-4C of FIG. 4B.

FIG. 5A is a high-level perspective view of an exemplary delivery assembly comprising a prosthetic valve crimped over a deflated balloon of the delivery apparatus of FIG. 3A.

FIG. 5B is a high-level perspective view of the delivery assembly of FIG. 5A, with the prosthetic valve shown in the expanded state over the inflated balloon.

FIG. 6A is a high-level perspective view of an exemplary delivery apparatus equipped with an axially movable ultrasound sensor positioned proximal to the balloon intermediate section.

FIG. 6B shows the delivery apparatus of FIG. 6A with the ultrasound sensor axially positioned within the balloon intermediate section.

FIG. 7A is a cross-sectional view of a distal portion of an exemplary delivery assembly, with the ultrasound sensor, attached to a spring and a pull-wire, shown in a first position.

FIG. 7B is a cross-sectional view of the delivery assembly of FIG. 7A with the ultrasound sensor moved to the second position.

FIG. 8A shows an exemplary bead-shaped stopper.

FIG. 8B shows an exemplary wedge-shaped stopper.

FIG. 9A is an enlarged perspective view of an exemplary ultrasound sensor disposed around a carrier.

FIG. 9B is a cross sectional view across line 9B-9B of FIG. 9A.

FIG. 10A is a cross-sectional view of a distal portion of an exemplary delivery assembly equipped with two ultrasound sensors, shown in a first position.

FIG. 10B is a cross-sectional view of the delivery assembly of FIG. 10A with the ultrasound sensors moved to the second position.

FIG. 11A is a cross-sectional view of a distal portion of an exemplary delivery assembly, with a spring disposed between the ultrasound sensor and a sensor shaft, shown in a first position.

FIG. 11B is a cross-sectional view of the delivery assembly of FIG. 11A with the ultrasound sensor moved to the second position.

FIG. 12 shows a prosthetic valve crimped over a balloon inside a crimping device.

FIG. 13A is a cross-sectional view of a distal portion of an exemplary delivery assembly, with a sleeve proximally extending from the ultrasound sensor, shown in a first position.

FIG. 13B is a cross-sectional view of the delivery assembly of FIG. 13A with the ultrasound sensor moved to the second position.

FIG. 14A is a cross-sectional view of a distal portion of an exemplary delivery assembly, with an ultrasound sensor wedged against the outflow end of a crimped prosthetic valve, shown in a first position.

FIG. 14B is a cross-sectional view of the delivery assembly of FIG. 14A with the ultrasound sensor moved to the second position.

FIG. 15A is a cross-sectional view of a distal portion of an exemplary delivery assembly, with an ultrasound sensor attached to a pull-wire, shown in a first position.

FIG. 15B is a cross-sectional view of the delivery assembly of FIG. 15A with the ultrasound sensor moved to the second position.

FIG. 16A is a cross-sectional view of a distal portion of an exemplary delivery assembly, with an ultrasound sensor residing, in a first position, inside a funnel-shaped portion of a nosecone proximal extension.

FIG. 16B is a cross-sectional view of the delivery assembly of FIG. 16A with the ultrasound sensor moved to the second position.

FIG. 17A is a cross-sectional view of a distal portion of an exemplary delivery assembly, with an ultrasound sensor attached to a sensor shaft, shown in a first position.

FIG. 17B is a cross-sectional view of the delivery assembly of FIG. 17A with the ultrasound sensor moved to the second position.

FIG. 18A is a perspective cut-away view of a distal portion of an exemplary delivery assembly with an exemplary ultrasound sensor and temperature sensor disposed inside a balloon thereof.

FIG. 18B is an enlarged perspective view of the ultrasound sensor and temperature sensor of FIG. 18A.

FIG. 19 is a perspective cut-away view of a distal portion of a delivery apparatus with an exemplary ultrasound sensor and temperature sensor extending from a dedicated shaft.

FIG. 20 is a perspective view of a delivery apparatus with an exemplary temperature sensor at a proximal portion thereof.

FIG. 21 is a perspective view of an exemplary delivery apparatus equipped with a pair of ultrasound sensors within a balloon thereof.

FIG. 22 is a perspective cut-away view of a distal portion of a delivery apparatus with an exemplary pair ultrasound sensors disposed around carriers.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a”, “an”, and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner”, “outer”, “upper”, “lower”, “inside”, “outside”, “top”, “bottom”, “interior”, “exterior”, “left”, right”, and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus. The terms “longitudinal” and “axial” are interchangeable, and refer to an axis extending in the proximal and distal directions, unless otherwise expressly defined.

The terms “axial direction”, “radial direction”, and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first”, “second”, and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a”, “b”, “c”, etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

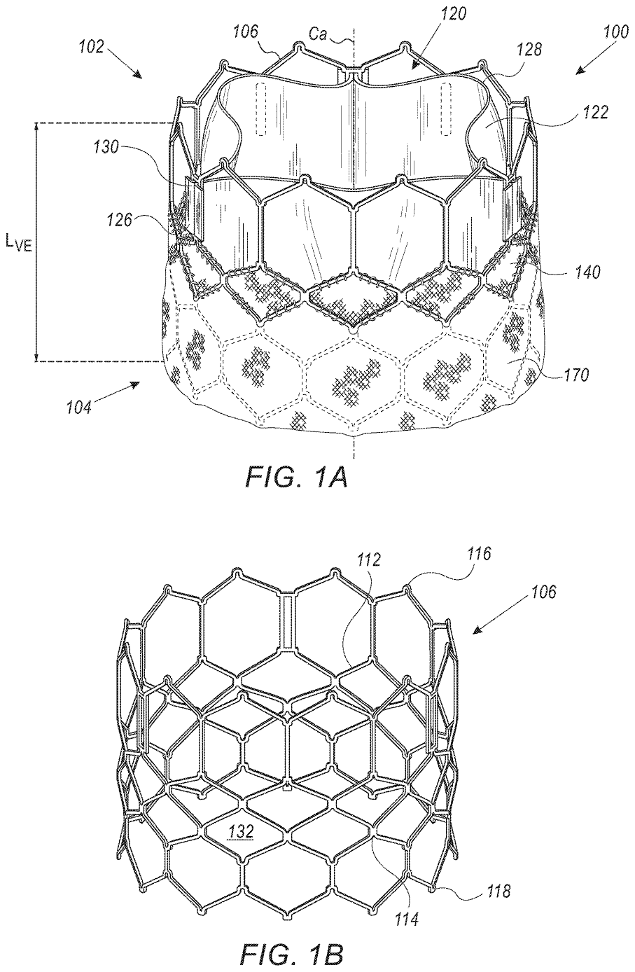

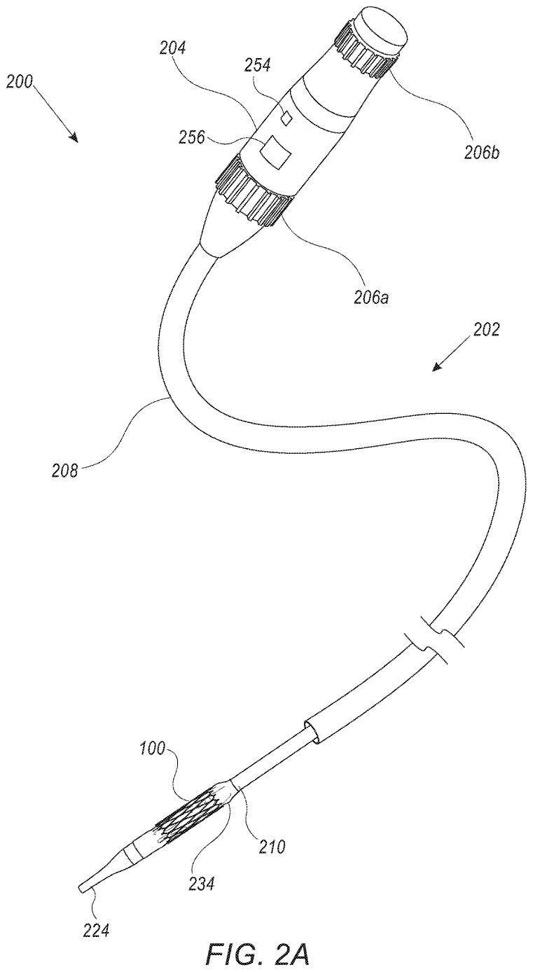

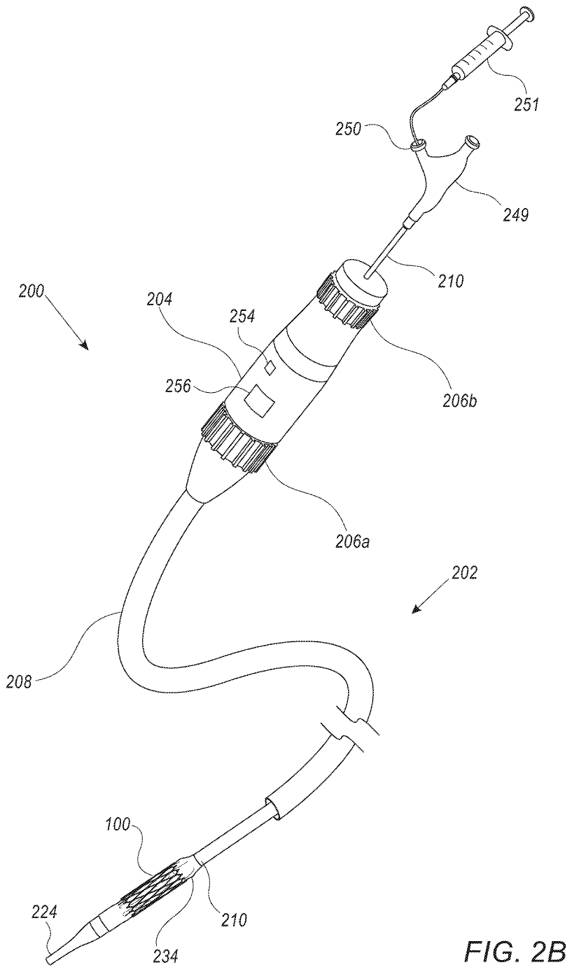

FIGS. 1A and 1B show perspective views of an exemplary prosthetic valve 100 with and without soft components attached thereto, respectively. FIG. 2A shows a perspective view of an exemplary delivery assembly 200. The delivery assembly 200 can include the prosthetic valve 100 and a delivery apparatus 202. The prosthetic valve 100 can be on or releasably coupled to the delivery apparatus 202.

The term “prosthetic valve”, as used herein, refers to any type of a balloon-expandable prosthetic valve deliverable to a patient's target site over a catheter, which is radially expandable and compressible between a radially compressed, or crimped, state, and a radially expanded state. Thus, a prosthetic valve 100 can be crimped or retained by a delivery apparatus 202 in a compressed state during delivery, and then expanded to the expanded state once the prosthetic valve 100 reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state.

A prosthetic valve 100 of the current disclosure may include any balloon-expandable prosthetic valve configured to be mounted within the native aortic valve, the native mitral valve, the native pulmonary valve, and the native tricuspid valve. While a delivery assembly 200 described in the current disclosure, includes a delivery apparatus 202 and a balloon expandable prosthetic device, such as prosthetic valve 100, it should be understood that the delivery apparatus 202 according to any example of the current disclosure can be used for implantation of other prosthetic devices aside from prosthetic valves, such as stents or grafts, and that delivery apparatus 202 can be used to deliver an inflatable balloon 234 that does not necessarily carry an expandable prosthetic device, for example for medical procedures that can include valvuloplasty, pre-ballooning and post-ballooning.

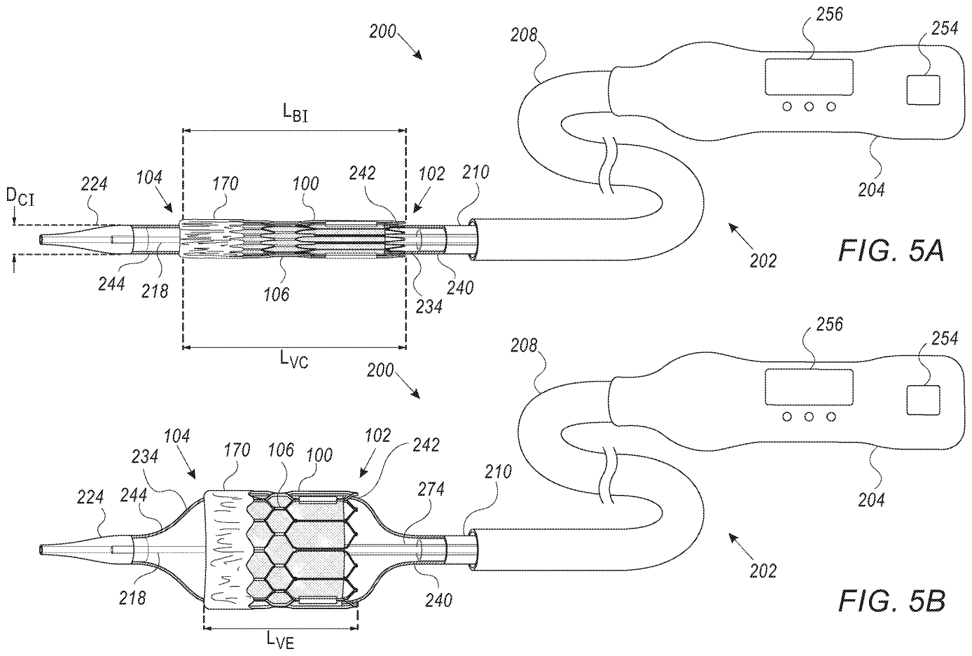

A catheter deliverable prosthetic valve 100 can be delivered to the site of implantation via the delivery assembly carrying the valve 100 in a radially compressed or crimped state, toward the target site, to be mounted against the native anatomy, by expanding the prosthetic valve 100. Balloon expandable valves 100 generally involve a procedure of inflating a balloon 234 within a prosthetic valve 100, thereby expanding the prosthetic valve 100 within the desired implantation site. Once the valve is sufficiently expanded, the balloon 234 is deflated and retrieved along with the delivery apparatus 202. A length of the prosthetic valve 100 can be defined between the inflow end 104 and the outflow end 102 in the axial direction, and can be shorter in the expanded state of the valve 100 relative to the crimped state due to valve foreshortening. Thus, the prosthetic valve 100 can have a valve crimped length L_{VC} as indicated, for example, in FIG. 5A, and a valve expanded length L_{VE} as indicated, for example, in FIGS. 1A and 5B, such that L_{VE}<L_{VC}.

The delivery assembly 200 can be utilized, for example, to deliver a prosthetic aortic valve for mounting against the aortic annulus, to deliver a prosthetic mitral valve for mounting against the mitral annulus, or to deliver a prosthetic valve for mounting against any other native annulus.

FIGS. 1A-1B show an example of a balloon expandable prosthetic valve 100, illustrated in an expanded state. The prosthetic valve 100 can comprise an outflow end 102, an inflow end 104, and a central longitudinal axis Ca extending in a direction from the inflow end 104 to the outflow end 102. In some instances, the outflow end 102 is the proximal end of the prosthetic valve 100, and the inflow end 104 is the distal end of the prosthetic valve 100. Nevertheless, depending for example on the delivery approach of the valve, the outflow end can be the distal end of the prosthetic valve, and the inflow end can be the proximal end of the prosthetic valve.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

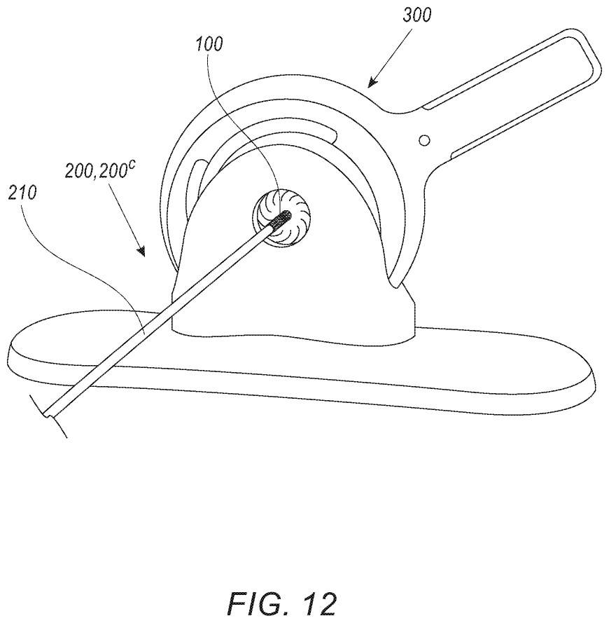

The prosthetic valve 100 comprises an annular frame 106 movable between a radially compressed configuration and a radially expanded configuration, and a leaflet assembly 120 mounted within the frame 106. The frame 106 can be made of various suitable materials, including plastically-deformable materials such as, but not limited to, stainless steel, a nickel-based alloy (e.g., a cobalt-chromium or a nickel-cobalt-chromium alloy such as MP35N alloy), polymers, or combinations thereof. When constructed of a plastically-deformable materials, the frame 106 can be crimped to a radially compressed state on a balloon catheter 210, for example by using a crimping device 300 (see FIG. 12), and then expanded inside a patient by an inflatable balloon 234.

In the example illustrated in FIGS. 1A-1B, the frame 106 is an annular, stent-like structure comprising a plurality of intersecting struts 112. In the current disclosure, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 112 may be any elongated member or portion of the frame 106. The frame 106 can include a plurality of strut rungs that can collectively define one or more rows of cells 132. The frame 106 can have a cylindrical or substantially cylindrical shape having a constant diameter from the inflow end 104 to the outflow end 102 as shown, or the frame can vary in diameter along the height of the frame, as disclosed in U.S. Pat. No. 9,155,619, which is incorporated herein by reference.

The end portions of the struts 112 are forming apices 116 at the outflow end 102 and apices 118 at the inflow end 104. The struts 112 can intersect at additional junctions 114 formed between the outflow apices 116 and the inflow apices 118. The junctions 114 can be equally or unequally spaced apart from each other, and/or from the apices 116, 118, between the outflow end 102 and the inflow end 104.

At least some of the struts 112 can be pivotable or bendable relative to each other, so as to permit frame expansion or compression. For example, the frame 106 can be formed from a single piece of material, such as a metal tube, via various processes such as, but not limited to, laser cutting, electroforming, and/or physical vapor deposition, while retaining the ability to collapse/expand radially in the absence of hinges and the like.

A leaflet assembly 120 of the prosthetic valve 100 can include a plurality of leaflets 122 (e.g., three leaflets), positioned at least partially within the frame 106, and configured to regulate flow of blood through the prosthetic valve 100 from the inflow end 104 to the outflow end 102. While three leaflets 122 arranged to collapse in a tricuspid arrangement, are shown in the example illustrated in FIG. 1A, it will be clear that a prosthetic valve 100 can include any other number of leaflets 122.

The inflow or cusp edges (concealed from view in FIG. 1A) of the leaflets 122 can be secured to the frame 106 directly or indirectly, such as by being sutured directly to the frame, being sutured to an inner skirt, and/or via one or more connecting skirts. The cusp portions of the leaflets 122 can collectively define a scalloped line of attachment. Further examples and methods of attaching skirts and seal members to a frame, as well as method and techniques for coupling leaflets 122 to the frame 106, with or without connecting skirts, are disclosed in U.S. Pat. No. 11,096,781, which is incorporated herein by reference.

Adjacent leaflets 122 can be arranged together to form commissures 130 that are coupled (directly or indirectly) to respective portions of the frame 106, thereby securing an upper portion (e.g., above the scalloped line) of the leaflet assembly 120 to the frame 106. In some examples, each leaflet 122 can comprise opposing tabs 126. Each tab 126 can be secured to an adjacent tab 126 of an adjacent leaflet 122 to form a commissure 130 that is secured to the frame 106. The tabs 126 can be folded in various manners, for example to form radially extending layers and circumferentially extending layers facing the frame. Radially extending layers can extends radially inward from a location on the frame 106 to free edges 128, also termed coaptation edges, of the leaflets.

During valve cycling, the leaflets 122 can articulate at the inner most edges of the tab layers, which helps space the leaflets away from the frame 106 during normal operation of the prosthetic valve. This is particular advantageous in cases where the prosthetic valve 100 is not fully expanded to its nominal size when implanted in a patient. As such, the prosthetic valve 100 can be implanted in a wider range of patient annulus sizes. Further details regarding transcatheter prosthetic valves, including the manner in which leaflets 122 can be coupled to the frame 106 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can further comprise at least one skirt or sealing member. In some examples, an inner skirt 140 can be secured to the inner surface of the frame 106, configured to function, for example, as a sealing member to prevent or decrease perivalvular leakage. An inner skirt 140 can further function as an anchoring region for the leaflets 122 to the frame 106, and/or function to protect the leaflets 122 against damage which may be caused by contact with the frame 106, for example during valve crimping or during working cycles of the prosthetic valve 100. In some examples, the prosthetic valve 100 can comprise an outer skirt 170 mounted on the outer surface of the frame 106, configure to function, for example, as a sealing member retained between the frame 106 and the surrounding tissue of the native annulus against which the prosthetic valve is mounted, thereby reducing risk of paravalvular leakage (PVL) past the prosthetic valve 100.

Any of the inner skirt 140 and/or outer skirt 170 can be made of various suitable biocompatible materials, such as, but not limited to, various synthetic materials (e.g., PET) or natural tissue (e.g., pericardial tissue). In some examples, the inner skirt 140 can be formed of a single sheet of material that extends continuously around the inner surface of frame 106. In some examples, the outer skirt 170 can be formed of a single sheet of material that extends continuously around the outer surface of frame 106.

FIG. 2A illustrates a delivery assembly 200 that includes a delivery apparatus 202, optionally adapted to deliver a prosthetic device, which can be the prosthetic valve 100 described above with respect to FIGS. 1A-1B. The delivery apparatus 202 includes a handle 204 and a balloon catheter 210 having an inflatable balloon 234 mounted on its distal end. A balloon expandable prosthetic device, such as balloon expandable prosthetic valve 100, can be carried in a crimped state over the balloon catheter 210.

In some examples, a delivery apparatus 202 further comprises an outer delivery shaft 208. Optionally, an outer delivery shaft 208 of a delivery apparatus 202 can concentrically extend over the balloon catheter 210.

The outer delivery shaft 208 and the balloon catheter 210 can be configured to be axially movable relative to each other. For example, a proximally oriented movement of the outer delivery shaft 208 relative to the balloon catheter 210, or a distally oriented movement of the balloon catheter 210 relative to the outer delivery shaft 208, can expose the prosthetic valve 100 from the outer delivery shaft 208.

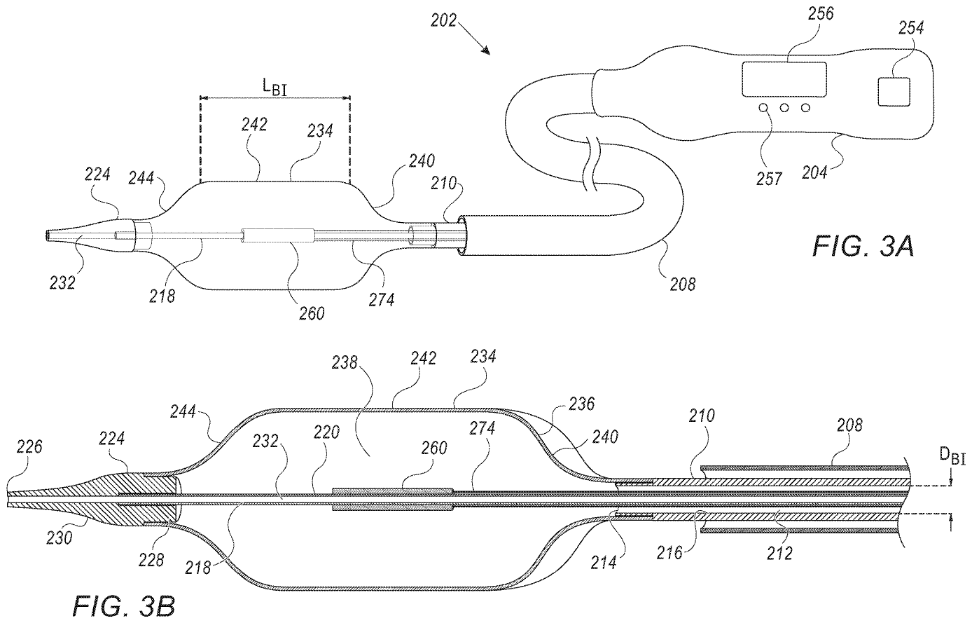

A delivery apparatus 202 can further include a nosecone 224 to facilitate advancement of the delivery apparatus 202 through the patient's vasculature to the site of treatment. A nosecone shaft 218 (shown, for example, in FIGS. 3A-3B) can extend proximally from the nosecone 224 through a lumen 212 of the balloon catheter 210. The nosecone 224 can be conical or frustoconical in shape, and can include a nosecone tapering outer surface 230 terminating at a nosecone distal end 226, and a nosecone proximal end portion 228 that can be coupled to the nosecone shaft 218 extending proximally therefrom. Attachment of the nosecone shaft 218 to the nosecone proximal end portion 228 can be achieved by a variety of methods, such as overmolding, radio-frequency welding, through an adhesive, and/or a combination thereof. In some examples (not illustrated), the nosecone shaft 218 can extend through the length of the nosecone 224, such that a distal end of the nosecone shaft 218 is aligned with the nosecone distal end 226. In some examples (not illustrated), the nosecone shaft 218 is coupled to one or more components, such as collars or other connectors, which are in turn attached to the nosecone 224.

In FIG. 2A, a prosthetic valve 100 is mounted on the balloon 234 and is shown in a crimped state, providing prosthetic valve 100 with a reduced diameter for delivery to the heart via the patient's vasculature. As mentioned above, it should be understood that balloon 234 can be configured for delivery to a treatment location without a prosthetic device (such as a prosthetic valve 100) mounted thereon, either for off-balloon delivery of the prosthetic device to a treatment location or for use of the balloon in a valvuloplasty procedure.

Although the illustrated examples discussed herein refer to the prosthetic device (e.g., prosthetic valve 100) as being crimped or mounted on the balloon 234 for delivery to the treatment location, it should be understood that the prosthetic device can be crimped or mounted at a location different from the location of balloon 234 (e.g., proximal to the balloon 234) and repositioned over the balloon at some time before inflating the balloon and deploying the prosthetic device. This off-balloon delivery allows the prosthetic device to be crimped to a lower profile than would be possible if the prosthetic device was crimped on top of the balloon 234. The lower profile permits the clinician to more easily navigate the delivery apparatus (including the crimped prosthetic device) through a patient's vasculature to the treatment location. The lower profile of the crimped prosthetic device can be particularly helpful when navigating through portions of the patient's vasculature which are particularly narrow, such as the iliac artery.

The proximal ends of the balloon catheter 210, the outer delivery shaft 208, and/or the nosecone shaft 218, can be coupled to the handle 204. During delivery, the handle 204 can be maneuvered by an operator (e.g., a clinician or a surgeon) to axially advance or retract components of the delivery apparatus 202, such as the nosecone shaft 218, the outer delivery shaft 208, and/or the balloon catheter 210, through the patient's vasculature and/or along the target site of implantation, as well as to inflate the balloon 234 mounted on the balloon catheter 210, for example to expand a prosthetic valve 100 mounted on the balloon 234, and to deflate the balloon 234 and retract the delivery apparatus 202, for example once the prosthetic valve 100 is mounted in the implantation site.

The handle 204 can include a steering mechanism configured to adjust the curvature of a distal end portion of the delivery apparatus 202. In the illustrated example, the handle 204 includes an adjustment member, such as the illustrated rotatable knob 206a, which in turn is operatively coupled to the proximal end portion of a pull wire (not shown). The pull wire can extend distally from the handle 204 through the outer delivery shaft 208 and has a distal end portion affixed to the outer delivery shaft 208 at or near the distal end of the outer delivery shaft 208. Rotating the knob 206a can increase or decrease the tension in the pull wire, thereby adjusting the curvature of the distal end portion of the delivery apparatus 202. Further details on steering or flex mechanisms for the delivery apparatus can be found in U.S. Pat. No. 9,339,384, which is incorporated by reference herein. The handle 204 can further include an adjustment mechanism including an adjustment member, such as the illustrated rotatable knob 206b. The adjustment mechanism can be configured to adjust the axial position of an ultrasound sensor of the delivery apparatus 202, as will be described in greater detail below. The handle can include additional knobs to control additional components of the delivery apparatus 202, such as positioning members that will be described in greater detail below.

A prosthetic valve 100 can be carried by the delivery apparatus 202 during delivery in a crimped state, and expanded, for example by balloon inflation, to secure it in a native heart valve annulus (such as an aortic annulus) or against a previously implanted prosthetic valve (for example, during valve-in-valve implantation procedures).

The balloon 234 can be secured to balloon catheter 210 at the balloon's proximal end, and to either the balloon catheter 210, the nosecone shaft 218, or the nosecone 224, at its distal end. In some examples, the balloon 234 is secured to a distal end portion of the balloon catheter 210 at its proximal end, while the balloon's distal end can be coupled, directly or indirectly, to another component of the delivery apparatus 202, such as the nosecone 224 or nosecone shaft 218.

In the examples illustrated in FIGS. 3A-3B, for example, the balloon 234 is shown to be coupled to the nosecone proximal end portion 228. The nosecone proximal end portion 228 can optionally include an outer step configured to accommodate the distal end of the balloon 234, such that the outer surface of the balloon 234 can be flush or otherwise relatively continuous with the outer surface of the nosecone 224. Similarly, in some examples, the distal end portion of the balloon catheter 210 can include an outer step configured to accommodate the proximal end of the balloon 234, such that the outer surface of the balloon 234 can be flush or otherwise relatively continuous with the outer surface of the balloon catheter 210.

In some examples, such as when the balloon 234 is attached at both ends thereof to the nosecone 224 and balloon catheter 210, both the nosecone 224 with nosecone shaft 218 and the balloon catheter 210 can be configured to move simultaneously in the axial direction, without necessarily being axially movable relative to each other, or while axial movement of one relative to the other is limited. In such examples, the delivery apparatus 202 can be designed such that axial movement of the balloon catheter 210 causes the nosecone shaft 218 to move therewith, or such that axial movement of the nosecone shaft 218 causes the balloon catheter 210 to move therewith.

Balloon 234 is configured to transition between a deflated state, shown for example in FIGS. 2 and 5A, and an inflated state, shown for example in FIGS. 3A-4A and 5B. When reaching the site of implantation, the deflated balloon 234, carrying crimped prosthetic valve 100 thereover, can be advanced to the target site to expand the prosthetic valve. Once the prosthetic valve 100 is expanded to its functional diameter within a native annulus or within a previously implanted prosthetic valve, the balloon 234 can be deflated, and the delivery apparatus 202 can be retrieved from the patient's body.

Balloon 234 comprises a balloon proximal section 240, a balloon distal section 244, and a balloon intermediate section 242 extending therebetween. The balloon proximal section 240 extends from a proximal end of the balloon 234, at which it can be attached to the balloon catheter 210, to the balloon intermediate section 242, and can include a portion configured to assume a proximally-tapering configuration in the inflated state of the balloon 234. The balloon distal section 244 extends from the balloon intermediate section 242 to a distal end of the balloon 234, at which it can be attached to the nosecone 224 (or any other distal component of the delivery apparatus 202), and can include a portion configured to assume a distally-tapering configuration in the inflated state of the balloon 234. When a prosthetic valve 100 is mounted on balloon 234, it may be disposed around the balloon intermediate section 242, while the balloon proximal section 240 and balloon distal section 244 can remain uncovered by prosthetic valve 100. Thus, the length L_{BI} of the balloon intermediate section 242 can be equal to, or greater than, the length L_{VC} of the prosthetic valve 100 in its crimped state. When a balloon is inflated without having a prosthetic valve 100 disposed therearound, such as during valvuloplasty procedures, the balloon intermediate section 242 can be defined as a section of the balloon 234 configured to expand to a diameter sufficient to contact the surrounding native anatomy (e.g., native annulus or a blood vessel wall).

As shown, for example, in FIGS. 3A-3B, the balloon catheter 210 can define a balloon catheter lumen 212 having a balloon catheter lumen diameter D_{BI}, through which a guidewire (not shown) and one or more additional shafts of the delivery apparatus 202 can extend. The balloon catheter 210 can extend through the handle 204 and be fluidly connectable to a fluid source for inflating the balloon 234.

In some examples, as shown in FIG. 2B, the balloon catheter 210 can extend through the handle 204 and a proximal portion 249 which can be disposed proximally to the handle 204. The proximal portion 249 can be formed with a fluid passageway 250 that is fluidly connectable to a fluid source 251 for inflating the balloon. The fluid source 251 comprises an inflation fluid.

The fluid source 251 comprises an inflation fluid. The term “inflation fluid”, as used herein, means a fluid (e.g., saline, though other liquids or gas can be used) used for inflating the balloon 234. The inflation fluid source 251 is in fluid communication with the balloon catheter lumen 212, such that fluid from the fluid source 251 can flow through the balloon catheter lumen 212 into balloon 234 to inflate it.

As further shown, for example, in FIG. 3B, the nosecone shaft 218 and the nosecone 224 collectively define a guidewire passage lumen 232 extending along the length of the nosecone shaft 218 and nosecone 224, through which the guidewire can pass, such that the delivery apparatus 202 can be advanced toward the treatment region over the guidewire. The outer diameter of the nosecone shaft 218 can be sized such that an annular space is formed within balloon catheter lumen 212 between balloon catheter inner surface 216 and the nosecone shaft outer surface 220 along the length of balloon catheter 210. This annular space can be in fluid communication with one or more inflation openings 214 exposed to an internal cavity 238 of the balloon 234, which can be in fluid communication with a fluid source (e.g., a syringe or a pump) that can inject an inflation fluid (e.g., saline) into balloon cavity 238. In this way, inflation fluid from the fluid source can flow through the balloon catheter lumen 212 into balloon cavity 238 via inflation opening(s) 214 to inflate the balloon 234, and optionally expand and deploy a prosthetic valve 100 when such a device is disposed thereon.

For example, the pressure of the fluid within balloon 234 may provide the force that allows the intermediate section 242 of balloon 234 to dilate the prosthetic valve 100 and/or surrounding anatomy. Further, the balloon catheter lumen 212 may be configured to withdraw fluid from the balloon cavity 238 through the opening(s) 214 to deflate the balloon 234. Thus, the balloon catheter lumen 212 may be utilized to inflate the balloon 234 to transition the balloon 234 to the inflated or deployed state, and may be utilized to deflate the balloon 234 to transition the balloon 234 to the deflated or undeployed state.

Nosecone shaft 218, balloon catheter 210, and optional outer delivery shaft 208, can be formed from, or include, any of various suitable materials, such as nylon, braided stainless steel wires, or a polyether block amide (commercially available as Pebax®). In some examples, balloon catheter 210 and/or optional outer delivery shaft 208 have longitudinal sections formed from, or including, different materials, in order to vary the flexibility of the shafts along their lengths. In some examples, nosecone shaft 218 has an inner liner or layer formed of Teflon® to minimize sliding friction with a guidewire. Balloon 234 includes a balloon wall 236 surrounding and defining the balloon cavity 238, which may be made of one polymer, or use several layers or a mix of different polymers. Polymers such as Nylon, PEBAX, PET, parylene and/or polyurethane may be used to make the balloon wall 236.

In some examples, any delivery assembly of the current disclosure can be packaged in a sterile package that can be supplied to end users for storage and eventual use. In some examples, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the delivery assembly can be free of any liquid. Methods for treating tissue leaflets for dry storage are disclosed in U.S. Pat. Nos. 8,007,992 and 8,357,387, both of which documents are incorporated herein by reference.

When inflating a balloon in valvuloplasty procedure, it is desirable to avoid applying excessive force to calcifications present in the artery and/or annulus. Additionally, when implanting a prosthetic device, such as balloon expandable prosthetic valve 100, it is desirable to expand the valve to a maximum size allowed by the patient's anatomical considerations, in order to avoid paravalvular leakage or other unfavorable hemodynamic phenomena across the valve that may be associated with a mismatch between the valve's expansion diameter and the surrounding tissue, while mitigating the risk of annular rupture that may result from over-expansion.

An exemplary delivery apparatus 202 can include, in some examples, at least one ultrasound sensor configured to measure a distance that can be indicative, in some examples, or a radius or diameter or any one of: a balloon 234, a prosthetic valve 100, and/or a lumen of a native anatomical structure in a patient's body. FIG. 3A illustrates a high-level perspective view of an exemplary delivery apparatus 202 equipped with an ultrasound sensor 260. FIG. 3B illustrates a high-level perspective cut-away view of a portion of the delivery apparatus 202 of FIG. 3A.

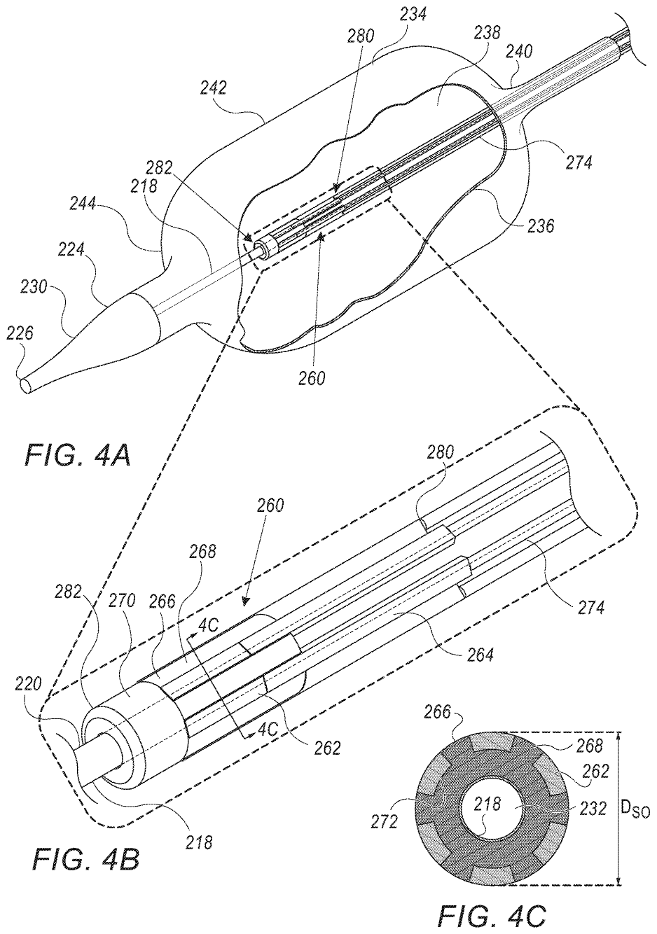

FIG. 4A shows a perspective cut-away view of a distal portion of a delivery apparatus 202 with an exemplary ultrasound sensor 260 disposed inside the balloon cavity 238. FIG. 4B shows an enlarged perspective view of the ultrasound sensor 260 of FIG. 4A. FIG. 4C shows a cross sectional view across line 4C-4C of FIG. 4B. FIG. 5A illustrates a high-level perspective view of an exemplary delivery assembly 200 comprising a prosthetic valve 100 crimped over a deflated balloon 234 of the delivery apparatus 202 of FIG. 3A. FIG. 5B illustrates a high-level perspective view of the delivery assembly 200 of FIG. 5A with the prosthetic valve 100 shown in the expanded state over the inflated balloon 234.

In some examples, the delivery apparatus 202 comprises a sensor data unit 254. The sensor data unit 254 can include, in some examples, a central processing unit (CPU), a microprocessor, a microcomputer, a programmable logic controller, an application-specific integrated circuit (ASIC) and/or a field-programmable gate array (FPGA), without limitation. In some examples, sensor data unit 254 comprises electrical circuitry.

In some examples, the ultrasound sensor 260 is in communication with the sensor data unit 254. In some examples, the ultrasound sensor 260 is in communication with the sensor data unit 254 via one or more optional communication line(s) 274. In some examples, the ultrasound sensor 260 is in wireless communication with the sensor data unit 254. In some examples, the one or more ultrasound sensor 260 can include a plurality of ultrasound sensors 260, wherein each of the sensors 260 can be in communication with the sensor data unit 254 via one or more respective communication line(s) 274. In some examples, an ultrasound sensor 260 can include a plurality of ultrasound transducers 262, wherein each ultrasound transducers 262 can be in communication with the sensor data unit 254 via a respective communication line 274. In some examples, when a plurality of ultrasound sensors 260 are provided, and/or when an ultrasound sensor 260 comprises a plurality of ultrasound transducers 262, the operation of the ultrasound sensor(s) 260 and/or the ultrasound transducer(s) 262 can be multiplexed such that only one respective ultrasound sensor 260 and/or only one respective ultrasound transducer 262 outputs data each time.

It is to be understood that any reference to an “ultrasound sensor 260” throughout the disclosure, in the singular form, may similarly refer to a plurality of ultrasound sensors 260, unless stated otherwise. Similarly, any reference to a “communication line 274” throughout the disclosure, in the singular form, may similarly refer to a plurality of communication lines 274, unless stated otherwise.

In some examples, communication line 274 is configured to allow: electrical communication via a conductive material, such as a wire; and/or optical communication, e.g., via an optical fiber. In some examples, the communication line 274 can include an electrically insulated cover.

In some examples, the ultrasound sensor 260 is operated by the sensor data unit 254 such that the sensing of ultrasound sensor 260 is performed in cooperation with the sensor data unit 254. In some examples, the ultrasound sensor 260 comprises dedicated circuitry for operation and the sensor data unit 254 receives the measured data from the ultrasound sensor 260. In some examples, each ultrasound transducer 262 is attached to a PCB component 264. In some examples, the ultrasound sensor 260 is in wireless communication with an external computing device (not shown).

In some examples, the delivery apparatus 202 comprises one or more visual or auditory informative elements configured to provide visual or auditory information and/or feedback to a user or operator of delivery apparatus 202, such as a display 256, LED lights 257, speakers (not shown) and the like. It is to be understood that while a display 256 and LED lights 257 are shown together in some of the drawings, such as, for example, FIG. 3A, for illustrative purpose, and that a handle can include a display 256 without LED lights 257, can include LED lights 257 without a display 256, and can include any other visual or auditory feedback means or combinations thereof.

In some examples, as shown for example in FIGS. 4A-4C, an ultrasound sensor 260 comprises one or more ultrasound transducer(s) 262 attached to a separator 266. The separator can optionally include a non-conductive material, such as a polymeric material. When a plurality of ultrasound transducers 262 are provided, they can reside inside depressions 272 formed in the separator 266, and circumferentially spaced from each other by spacer portions 268 of the separator 266. In some examples, the separator 266 can include a separator distal portion 270 defining a ring-like structure distal to the ultrasound transducer(s) 262. As further shown in FIGS. 4A-4B, each ultrasound transducer 262 can be attached to a PCB component 264 extending proximally therefrom, with a communication line 274 extending proximally from each respective PCB component 264. In some examples, when an ultrasound sensor 260 includes a plurality of communication lines 274 attached to a plurality of ultrasound transducers 262 thereof, optionally via a plurality of corresponding PCB components 264, the plurality of communication lines 274 can be separated from each other along the path extending from the ultrasound sensor 260 towards the sensor data unit 254, can be grouped together.

While an ultrasound sensor 260 including six ultrasound transducers 262 is illustrated in FIGS. 4A-4C, it is to be understood that any other number, including a single ultrasound transducer, two to five ultrasound transducers, or more than six ultrasound transducers, is contemplated. In one example, an ultrasound sensor 260 comprises at least two ultrasound transducers 262, the orientation of a first ultrasound transducer 262 opposing the orientation of the second ultrasound transducer 262. Since the ultrasound sensor 260 may be radially offset inside a balloon cavity 238, for example by being closer to one side of the balloon 234 in the radial direction, during and/or after balloon inflation, diametrically opposite ultrasound transducer 262 can measure the radial distance to each side, such that the total diameter can be estimated based on both radial distances. In some examples, where each ultrasound transducer 262 exhibits a 180-degree scanning range, a first ultrasound transducer 262 of two diametrically ultrasound transducers 262 can be oriented to cover a predetermined 180 degrees of the circumference of the balloon wall 236, and the second ultrasound transducer 262 can be oriented to cover the other 180 degrees of the circumference of the balloon wall 236.

In some examples, more than two ultrasound transducers are provided, such as the six ultrasound transducers 262 shown in FIGS. 4A-4C. For example, an array of ultrasound transducers can span circumferentially (e.g., around separator 266), configured to cover 360-degree scanning. More than six ultrasound transducers 262 can be provided in some examples to increase the resolution of the measurements. A plurality of ultrasound transducers 262 can be equally or not equally spaced from each other around the circumference of the separator 266.

The ultrasound sensor 260 defines a sensor outer diameter D_{SO}, indicated, for example, in FIG. 4C. In some examples, the sensor outer diameter D_{SO} is smaller than the balloon catheter inner diameter L_{BI}, to allow passage of the sensor 260 through the balloon catheter lumen 212.

In some examples, an ultrasound sensor 260 can be positioned inside the balloon cavity 238, as shown, for example, in FIGS. 3A-4A, such that one or more ultrasound transducer(s) 262 thereof faces an inner surface of balloon wall 236. In some examples, upon inflation of balloon 234, each ultrasound transducer 262 generates ultrasound waves and measures the waves reflected from a reflective surface, which can be an inner surface of the balloon wall 236 and/or a surface of a prosthetic valve 100 disposed around the balloon 234. Responsive to the measured waves, sensor data unit 254 can determine a diameter indication of the balloon 234 and/or the prosthetic valve 100 (such as the frame 106 of the prosthetic valve 100).

In some examples, the diameter indication is representative of a radial diameter of the balloon wall 236 and/or the prosthetic valve 100. In some examples, the diameter indication comprises the distance(s) between the respective ultrasound transducer(s) 262 and the balloon 234 and/or the prosthetic valve 100, i.e., a radial radius of the balloon wall 236 and/or the prosthetic valve 100. In some examples, the radial diameter comprises a sum of: the distance between a first ultrasound transducer 262 and the balloon wall 236 and/or the prosthetic valve 100; the distance between a second ultrasound transducer 262 and the balloon wall 236 and/or the prosthetic valve 100, respectively, wherein the orientation of the second ultrasound transducer 262 generally opposes the orientation of the first ultrasound transducer 262; and the sensor outer diameter D_{SO}, in order to include the space between the two ultrasound transducers 262.

As mentioned above, an ultrasound sensor 260 may not be exactly in the radial center of balloon 234, thus the distance between one ultrasound transducer 262 and the balloon wall 236 and/or the frame 106 of the prosthetic valve 100 may not give an exact radius of the respective balloon 234 and/or prosthetic valve 100. Advantageously, using the sum of the measurements of two ultrasound transducers 262 diametrically opposite to each other gives a more accurate measurement of the diameter of the balloon 234 and/or the prosthetic valve 100.

In some examples, responsive to an output of the ultrasound sensor 260, a diameter indication of the balloon 234 and/or prosthetic valve 100 is determined, and an indication of the determined diameter indication is generated, for example, via the screen 256 of the handle 204. In some examples, the sensor data unit 254 determines the diameter indication. The term “diameter indication”, as used herein, means a predetermined indication of a diameter. As mentioned above, the diameter indication can comprise the radial diameter of the balloon 234 and/or of the prosthetic valve 100. In some examples, the diameter indication is determined based at least in part on a predetermined function of the diameter. In some examples, the diameter indication comprises the amount of a change in the diameter. For example, sensor data unit 254 can determine the rate of change in the diameter of the balloon 234 and/or of the prosthetic valve 100, and/or the amount the diameter has changed between two or more measurements.

In some examples, upon detection that the diameter is no longer increasing (for example, when the balloon wall 236 and/or a prosthetic valve 100 disposed therearound is pushing against the walls of an anatomical wall at the site of treatment), sensor data unit 254 can output a signal indicating that the diameter is no longer increasing. In such a case, it may be desired to cease the injection of inflation fluid into the balloon 234, so as to reduce risk of damaging the surrounding tissue due to increased pressure applied by the balloon 234 and/or prosthetic valve 100 thereto. In some examples, sensor data unit 254 is further configured to control a flow controller (not shown) to prevent the flow of any more inflation fluid into the balloon 234.

In some examples, sensor data unit 254 outputs the determined diameter indication to a user display of handle 204, such as display 256. In some examples, sensor data unit 254 outputs the determined diameter indication to an external display or system (not shown). In some examples, responsive to the determined diameter indication, sensor data unit 254 outputs an indication of the maximum diameter allowed for expansion within the site of treatment. For example, balloon 234 can be used to determine the size of an anatomical lumen inside a patient, such as the size of the annulus, for purposes of determining the target expansion diameter of a prosthetic valve 100. In some examples, sensor data unit 254 can measure the diameter of a lumen inside the patient, for example during inflation of the balloon 234, thereby identifying the appropriate expansion diameter for a prosthetic valve 100.

During various medical procedures, such as pre-ballooning, post-ballooning, and/or prosthetic valve implantation, it may be desired to measure expansion diameter within a corresponding native annulus in which the balloon is inflated, to detect a mismatch between the expansion diameter of the balloon and/or prosthetic valve and the annulus. Since the balloon 234 extends through the annulus while being inflated during such procedures, the ultrasound sensor 260 is positioned at an appropriate axial position within the balloon cavity 238, such as at an appropriate axial position of the balloon intermediate section 242, for alignment with the native annulus. However, an ultrasound sensor 260 may have a non-negligible radial thickness, which can increase the crossing profile at the region of the sensor during delivery. The crossing profile, also termed the crimped profile when a prosthetic valve is crimped over the balloon, is important because it directly influences the clinician's ability to advance the delivery assembly 200 through blood vessels in the patient's vasculature. A smaller profile allows for treatment of a wider population of patients, with enhanced safety.

If the ultrasound sensor is axially affixed in position inside the balloon cavity 238, for example by being attached to the nosecone shaft 218 at an axial position along the balloon intermediate section 242, the sensor can significantly increase the crossing profile. When a prosthetic valve 100 is crimped around the balloon 234 during delivery, the added thickness of the ultrasound sensor 260 can significantly affect the crimped profile, especially if the sensor 260 is axially positioned to be closer to the level of the annulus during inflation, which is can be a relatively thick region of the prosthetic valve due to an outer skirt 170 extending from the inflow end 104 of the prosthetic valve 100 at the position configured to expand against the annulus, which may be, in some cases, a relatively puffy outer skirt 170 configured to improve PVL sealing, but also affecting the overall crimped profile at this region.

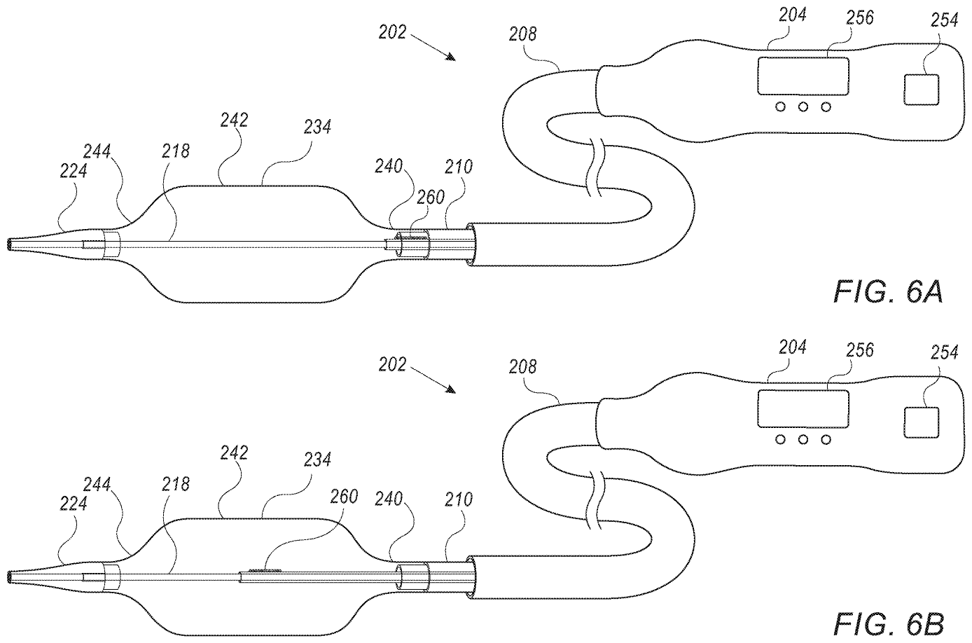

Disclosed herein are delivery assemblies 200 and delivery apparatuses 202 thereof, comprising one or more ultrasound sensor(s) 260 axially movable through the balloon cavity 238, between a first position axially offset from the balloon intermediate section 242, such as proximal or distal thereto, and a second position within the balloon intermediate section 242. FIG. 6A illustrates a high-level perspective view of an exemplary delivery apparatus 202 equipped with an axially movable ultrasound sensor 260 positioned proximal to the balloon intermediate section 242. FIG. 6B shows the delivery apparatus 202 of FIG. 6A with the ultrasound sensor 260 axially positioned within the balloon intermediate section 242. Some components of the delivery apparatus 202, such as communication lines 274, are removed from view for illustrative purpose.

During delivery of the apparatus 202 through the patient's vasculature, the ultrasound sensor 260 can be retained in the first position, for example, proximal to the balloon intermediate section 242 as shown in FIG. 6A. It is to be understood that the balloon 234 is shown both in FIGS. 6A and 6B in the inflated state for illustrative purpose, and that during delivery, the ultrasound sensor 260 can be axially offset from the balloon intermediate section 242 while the balloon 234 is in the deflated state. Upon reaching the site of treatment, and prior to and/or during balloon inflation, the ultrasound sensor 260 can be moved to the second position, as shown, for example, in FIG. 6B. Mechanism by which the ultrasound sensor 260 can move between the first and second positions can include passive or active movement mechanisms, as will be described in greater detail below. While a delivery apparatus 202 is illustrated without a prosthetic valve 100 disposed around the balloon 234, which can be applicable for some procedures, such as valvuloplasty, it is to be understood that a delivery assembly 200 having the delivery apparatus 202 described with respect to FIGS. 6A-6B can be further equipped with a prosthetic valve 100 disposed around the balloon 234.

Because ultrasound sensor 260 is positioned at a location axially offset from balloon intermediate section 242, prosthetic valve 100 can be crimped to a lower profile than would be possible if it was crimped on top of balloon 234 while the ultrasound sensor 260 is inside the balloon intermediate section 242, around which the prosthetic valve 100 is compressed. This lower profile permits the clinician to more easily navigate the delivery assembly 200 (including one or more axially movable ultrasound sensor(s) 260) through a patient's vasculature to the treatment location. The lower profile of the crimped prosthetic valve is particularly helpful when navigating through portions of the patient's vasculature which are particularly narrow, such as the iliac artery.

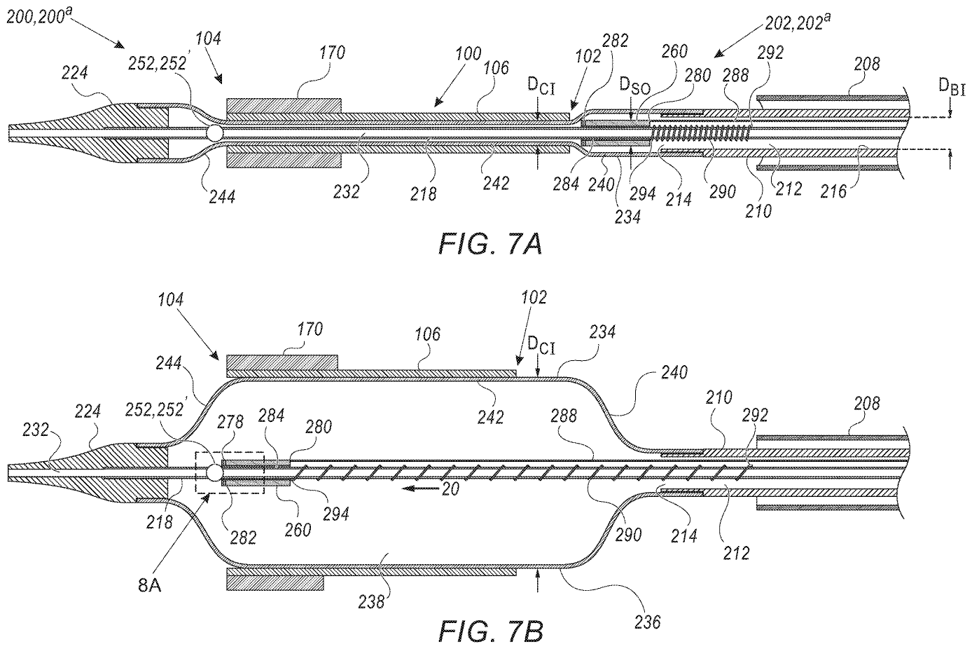

Various exemplary implementations for delivery assemblies 200 can be referred to, throughout the specification, with superscripts, for ease of explanation of features that refer to such exemplary implementations. It is to be understood, however, that any reference to structural or functional features of any apparatus, assembly or component, without a superscript, refers to these features being commonly shared by all specific exemplary implementations that can be also indicated by superscripts. In contrast, features emphasized with respect to an exemplary implementation of any apparatus, assembly or component, such as delivery assemblies 200 and/or delivery apparatuses 202 thereof, referred to with a superscript, may be optionally shared by some but not necessarily all other exemplary implementations. For example, delivery assembly 200^{a} shown in FIGS. 7A-7B is an exemplary implementation of delivery assembly 200, and thus includes features described for delivery assembly 200 throughout the current disclosure, except that while delivery assembly 200 comprises a delivery apparatus 202 having one or more ultrasound sensor(s) 260 that can be actively or passively moved between the first and second positions according to various movement mechanisms, delivery assembly 200^{a} includes a delivery apparatus 202^{a} comprising a spring-loaded ultrasound sensor 260 attached to a pull-wire 288.

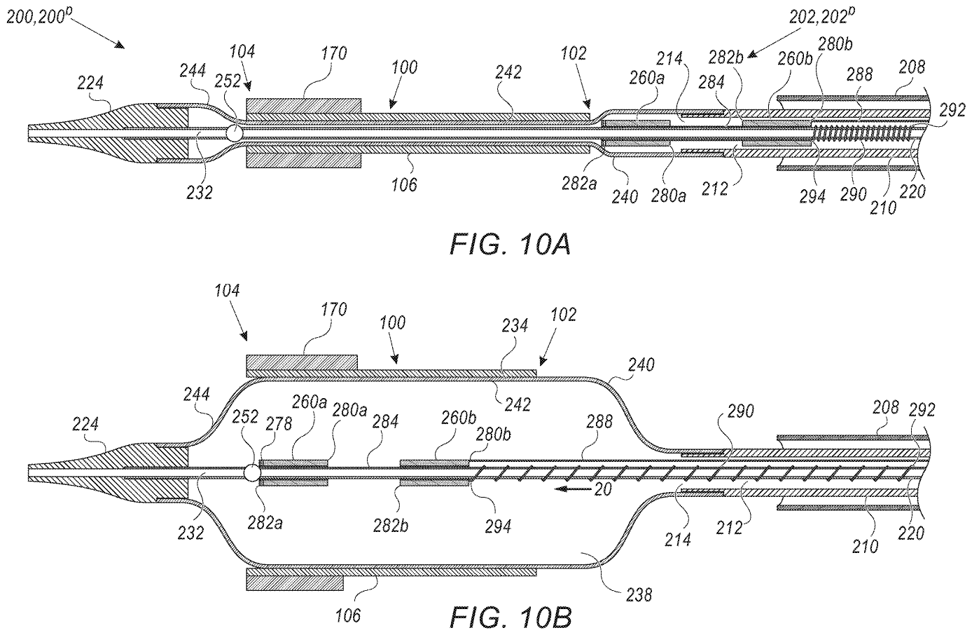

FIG. 7A is a cross-sectional view of a distal portion of an exemplary delivery assembly 200^{a} with the ultrasound sensor 260 shown in a first position while the balloon 234 is deflated and the prosthetic valve 100 is crimped thereover. FIG. 7B is a cross-sectional view of the delivery assembly 200^{a} with the balloon 234 inflated and the ultrasound sensor 260 moved to the second position. It is to be understood that some components of delivery assemblies 200 illustrated herein are removed from view from FIGS. 7A-7B, as well as FIGS. 10A-11B and 13A-17B, such as communication lines 274, for illustrative purpose.

Prosthetic valve 100 is schematically shown in FIGS. 7A-7B, 10A-11B and 13A-17B, to include a frame 106 disposed around the balloon 234 and an optional outer skirt 170 extending from the inflow end 104, though it is to be understood that an outer skirt 170 is merely optional, and that some types of prosthetic valves 100 can be provided without an outer skirt. Additional components of the prosthetic valve 100, such as the leaflet assembly 120 and an optional inner skirt 140, are omitted from view in these drawings for illustrative purpose. While delivery assemblies 200 are illustrated in FIGS. 7A-7B, 10A-11B and 13A-17B to include a prosthetic valve 100 mounted on balloon 234, it is to be understood that in some examples, the delivery assemblies 200 shown in these drawings can include a delivery apparatus 202 devoid of a prosthetic valve, for example when utilized for valvuloplasty, pre-ballooning, post-ballooning, and the like, unless stated otherwise.

In some examples, delivery apparatus 202^{a} comprises a spring 290 configured to bias the ultrasound sensor 260 towards the second position, in a free state of the spring 290 as shown in FIG. 7B. As shown, a distal end 294 of the spring 290 is attached to the ultrasound sensor 260 directly or indirectly, such as be being attached to a carrier 284 over which the sensor 260 is situated, and a proximal end 292 of the spring 290 is attached to an axially immovable component of the delivery apparatus 202^{a}, such as the nosecone shaft 218. A pull-wire 288 can be further attached to the sensor 260, such as at sensor proximal portion 280, and extend proximally from the sensor 260, through the balloon catheter lumen 212, towards the handle 204.

When a prosthetic valve 100 is crimped around the balloon intermediate section 242, it can compress the balloon intermediate section 242 to a diameter D_{CI} that can be smaller than the sensor outer diameter D_{SO}. FIG. 7A shows a state in which the sensor 260 is spring loaded, with the sensor distal portion 282 pressed against the portion of balloon wall 236 covering the outflow end 102 of the prosthetic valve 100, thus keeping the spring 290 in a compressed state, preventing distal advancement of the sensor 260 as long as the prosthetic valve 100 remains crimped over the balloon intermediate section 242. In the example illustrated in FIG. 7A, the sensor 260 is shown to be positioned proximal to the balloon intermediate section 242 in the first position, at the balloon proximal section 240. In some examples, the sensor 260 can be positioned inside the balloon catheter lumen 212, such as when the sensor outer diameter D_{SO} is less than the balloon catheter inner diameter D_{BI}.

When the balloon 234 is inflated, such as to expand the prosthetic valve 100, the sensor 260 is no longer axially blocked, allowing the spring 290 to extend to its free extended state, moving the sensor 260 in the distal direction 20 to the second position, as shown in FIG. 7B. In some examples, a delivery apparatus 202 can include a stopper 252 at a desired axial position within the balloon cavity 238, configured to stop further movement of the sensor 260, so as to position the sensor 260 in the desired second position. In some examples, the stopper is disposed around the nosecone shaft 218.

In the example illustrated in FIG. 7B, the stopper 252 is positioned at a distal end portion of the balloon intermediate section 242, or a proximal portion of the balloon distal section 244, in a manner that can result in the sensor 260 being axially closer to the inflow end 104 of the prosthetic valve 100 than the outflow end 102, in the second position. This can align the sensor, during a prosthetic valve implantation procedure, with the annulus in which the prosthetic valve 100 is to be implanted. It is to be understood that other axial positions of the stopper 252 are contemplated, so as to position the sensor 260 at any desired axial position along the balloon intermediate section 242 when moved to the second position.

The handle 204 can include a mechanism (not shown) for pulling the pull-wire 288, operable by a knob (such as knobs 206), so as to pull and/or release the pull-wire 288. In some examples, after reaching the second position as shown in FIG. 7B, an operator of the delivery apparatus 202^{a} can proximally pull the pull-wire 288 so as to compress the spring 290 and proximally move the sensor 260 from the second position to any desired axial position along the balloon intermediate section 242. This may allow for diameter measurements at different axial position that can be between the second and the first positions.

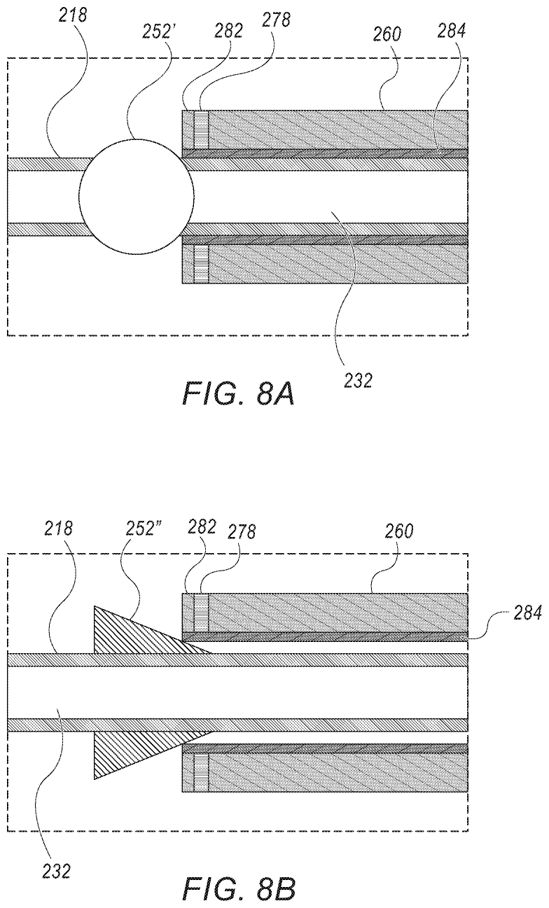

FIG. 8A is a zoomed in view of region 8A in FIG. 7B, showing one example of a stopper 252′ in the form of a spherical bead disposed around, and affixed to, the nosecone shaft 218. FIG. 8B shows another example of a wedge-shaped stopper 252″, having an outer surface tapering in the proximal direction, against which the sensor 260 and/or a carrier 284 thereof can be wedged. It is to be understood that other shapes and configurations are contemplated for a stopper 252, such as any type of protrusion affixed to, and extending radially away from, the outer surface of the nosecone shaft 218.

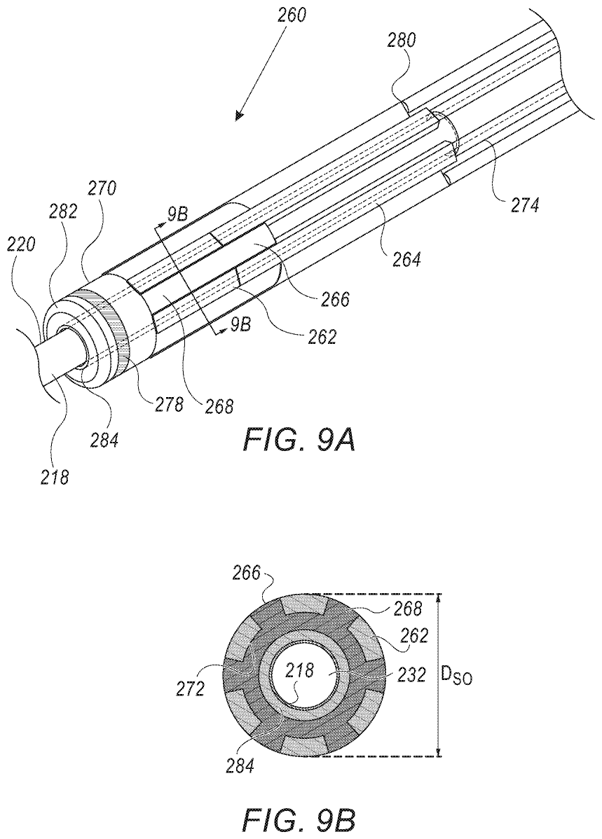

FIG. 9A shows an enlarged perspective view of an exemplary axially movable ultrasound sensor 260. FIG. 9B shows a cross sectional view across line 9B-9B of FIG. 9A. In some examples, an ultrasound sensor 260 can be attached to, and disposed around, a carrier 284. The carrier 284 defines an inner surface configured to axially slide along the nosecone shaft 218. In some examples, the carrier 284 is a tubular member that can be attached to the separator 266, as illustrated in FIGS. 9A-9B. In some examples, carrier 284 is integrally formed with the separator 266, defining an inner surface of the separator 266 surrounding the nosecone shaft 218.

In some examples, the ultrasound sensor 260 can include one or more radiopaque marker 278. In the example illustrated in FIG. 9A, a radiopaque marker 278 is shown around the separator distal portion 270. This can assist in identifying the position of the sensor 260, such as the optional position of the sensor distal portion 282, in the first and/or second position, under X-ray and/or fluoroscopic imagery. While a single radiopaque marker 278 is shown, it is to be understood that a plurality of radiopaque markers are contemplated, optionally axially spaced from each other. While a ring-shaped radiopaque marker 278 is illustrated, it is to be understood that any other shape is contemplated.

FIG. 10A is a cross-sectional view of a distal portion of an exemplary delivery assembly 200^{b} with the ultrasound sensor 260 shown in a first position while the balloon 234 is deflated and the prosthetic valve 100 is crimped thereover. FIG. 10B is a cross-sectional view of the delivery assembly 200^{b} with the balloon 234 inflated and the ultrasound sensor 260 moved to the second position. Delivery assembly 200^{b} is similar to any example described herein for delivery assembly 200^{a}, except that delivery apparatus 202^{b} of delivery assembly 200^{b} comprises a plurality of ultrasound sensors 260 (for example, a first sensor 260a and a second sensor 260b).

In the example illustrated in FIG. 10A-10B, two ultrasound sensors, such as a first or distal sensor 260a and a second or proximal sensor 260b, are axially spaced from each other and are both attached to a common elongated carrier 284. The distal end 294 of the spring 290 can be attached to the second sensor 260b and/or to a proximal end of the carrier 284. Other components and functionalities of the delivery assembly 200^{b} can be similar to those described above with respect to delivery assembly 200^{a}, and in the interest of brevity will not be discussed further.

When a plurality of ultrasound sensors 260 are provided, the sensor distal portion 282a of the first or distal-most sensor 260a can press against the portion of the balloon wall 236 covering the outflow end 102 of the crimped prosthetic valve 100 in the first position shown in FIG. 10A, and to contact the stopper 252 in the second position shown in FIG. 10B. In some examples, each of the plurality of ultrasound sensors 260 can include one or more radiopaque markers 278. While two ultrasound sensors 260 are shown in the illustrated example, it is to be understood that any other number of ultrasound sensors 260 is contemplated, including a series of more than two sensors 260. Inclusion of a plurality of ultrasound sensors 260 which are axially spaced from each other, can advantageously increase the resolution of the acquired data by enabling measurement of diameter at more than one axial position along the length of the balloon intermediate section 242 and/or along the length of the prosthetic valve 100.

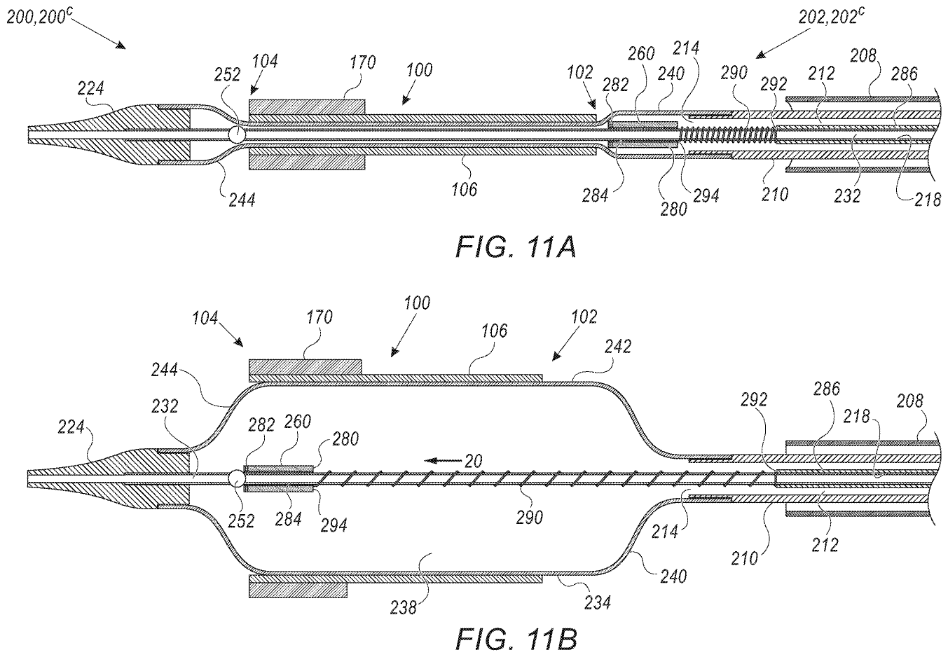

FIG. 11A is a cross-sectional view of a distal portion of an exemplary delivery assembly 200^{c} with the ultrasound sensor 260 shown in a first position while the balloon 234 is deflated and the prosthetic valve 100 is crimped thereover. FIG. 11B is a cross-sectional view of the delivery assembly 200^{c} with the balloon 234 inflated and the ultrasound sensor 260 moved to the second position. Delivery assembly 200^{c} is similar to any example described herein for delivery assemblies 200^{a} or 200^{b}, except that delivery apparatus 202^{c} of delivery assembly 200^{c} comprises a sensor shaft 286 and may be devoid of a pull-wire 288. The sensor shaft 286 extends through the balloon catheter lumen 212, optionally around the nosecone shaft 218, and is axially movable relative to the balloon catheter 210 and/or nosecone shaft 218.

The distal end 294 of the spring 290 is attached to the sensor 260, such as by direct attachment or by being indirectly attached to a carrier 284 of the sensor 260. The proximal end 292 of the spring 290 is attached to the sensor shaft 286, optionally to a distal end of the sensor shaft 286. In the first position of the sensor 260 illustrated in FIG. 11A, the distance between the sensor shaft 286 and the sensor 260 is shown to be short enough to compress the spring 290, while the sensor 260 is prevented from moving distally by the outflow end 102 of the crimped valve 100. When the balloon is inflated, such as to expand the prosthetic valve 100, the sensor 260 is no longer axially blocked, allowing the spring 290 to extend to its free extended state, moving the sensor 260 in the distal direction 20 to the second position, as shown in FIG. 11B. It is to be understood that while a single sensor 260 is illustrated in FIGS. 11A-11B, the delivery apparatus 202^{c} can include a plurality of ultrasound sensors 260, which can be coupled to a carrier 284 and implemented in a manner similar to that described with respect to FIGS. 10A-10B, mutatis mutandis.

The handle 204 can include a mechanism (not shown) for axially moving the sensor shaft 286, operable by a knob (such as knobs 206), so as to pull and/or push the sensor shaft 286. In some examples, after reaching the second position as shown in FIG. 11B, an operator of the delivery apparatus 202^{c} can proximally pull the sensor shaft 286 so as to proximally move the sensor 260 from the second position to any desired axial position along the balloon intermediate section 242. This may allow for diameter measurements at different axial position that can be between the second and the first positions.

Prior to insertion into the patient's body, a crimping device 300, as shown in FIG. 12, can be used to crimp the prosthetic valve 100 to the compressed configuration, which can be then stored in this configuration up to utilization thereof for implantation into the patient's body. As shown in FIG. 12, the prosthetic valve 100 can be inserted into a receiving opening of a crimping device 300 while the valve is placed over the deflated balloon 234. A crimping device 300 can be designed to translate a rotational movement of its housing to convergence of a plurality of plates thereof around the inner receiving opening into which the prosthetic valve 100 is inserted, thereby crimping the prosthetic valve 100 onto the balloon 234. In some examples, the crimping device 300 can include twelve plates configured to converge to crimp a prosthetic valve 100. Nevertheless, any other type of a crimping device 300 known in the art can be utilized for compressing the prosthetic valve 100.

In some examples, preloading the spring 290 of a delivery assembly 200^{a} or 200^{b} and placing the ultrasound sensor(s) 260 in the first position can be accomplished by pulling on the pull-wire 288 to pull the sensor(s) 260 against the spring 290, thereby compressing the spring 290, prior to crimping the valve 100. With the sensor(s) 260 maintained in a position proximal to the prosthetic valve 100, the valve 100 can be crimped over the balloon 234 inside the crimping device 300, after which the pull-wire 288 can be released, relying on the outflow end 102 of the crimped valve 100 being aligned against the sensor distal portion 282 to keep the sensor(s) 260 in the first position.

Similarly, in some examples, preloading the spring 290 of a delivery assembly 200^{c} and placing the ultrasound sensor(s) 260 in the first position can be accomplished by positioning the sensor 260 proximal to the prosthetic valve 100 prior to crimping, with the spring 290 optionally in an extended free state thereof. After crimping the valve 100 over the balloon 234, the sensor shaft 286 is distally advanced until the sensor 260 is stopped, in the first position, by the outflow end 102 of the prosthetic valve 100. Continued distal movement of the sensor shaft 286 serves to compress the spring 290, pre-loading it to the state shown in FIG. 11A for example.

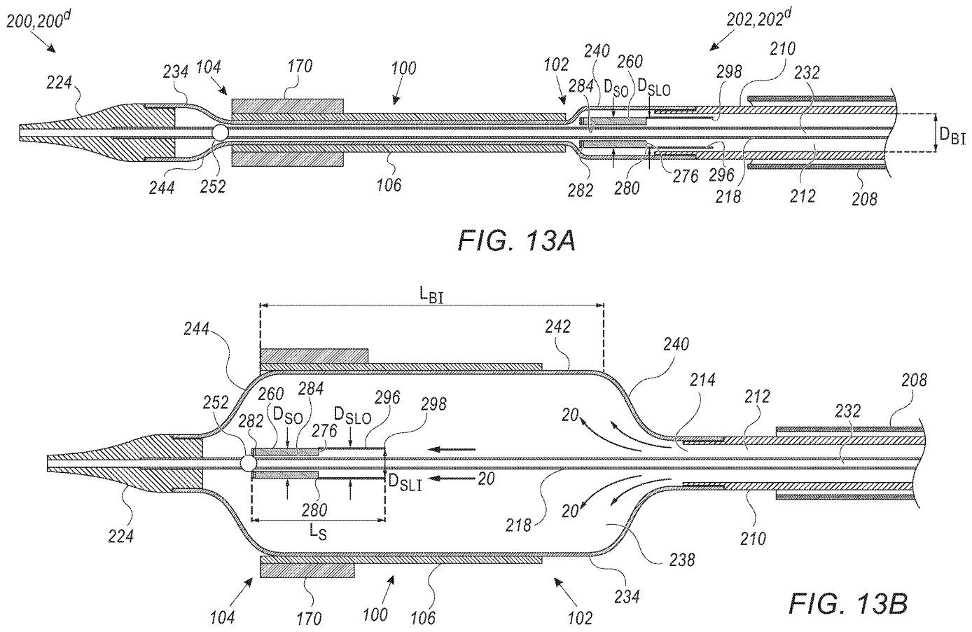

FIG. 13A is a cross-sectional view of a distal portion of an exemplary delivery assembly 200^{d} with the ultrasound sensor 260 shown in a first position while the balloon 234 is deflated and the prosthetic valve 100 is crimped thereover. FIG. 10B is a cross-sectional view of the delivery assembly 200^{d} with the balloon 234 inflated and the ultrasound sensor 260 moved to the second position. Delivery assembly 200^{d} is an exemplary implementation of delivery assembly 200, and thus can include any of the features described for delivery assembly 200 throughout the current disclosure, except that while delivery assembly 200 comprises a delivery apparatus 202 having one or more ultrasound sensor(s) 260 that can be actively or passively moved between the first and second positions according to various movement mechanisms, delivery assembly 200^{d} includes a delivery apparatus 202^{d} comprising a sleeve 296 extending proximally from the ultrasound sensor 260, configured to facilitate passive advancement of the sensor 260 by inflation fluid pushing the sensor 260 from the first to the second position during balloon inflation, as described in greater detail below.

The sleeve 296 can be attached to the ultrasound sensor 260, such as to the sensor proximal portion 280, and extend therefrom to terminate at a sleeve proximal end 298, defining a lumen between the sensor proximal portion 280 and the sleeve proximal end 298 which is exposed to the balloon catheter lumen 212 in the first position, as shown in FIG. 13A. In some examples, in the first position of the sensor 260, the sleeve 296 can extend into the balloon catheter lumen 212 such that the sleeve proximal end 298 may be positioned in close proximity to, or proximal to, the inflation opening(s) 214 at the distal end of the balloon catheter 210. The sleeve defines a sleeve inner diameter D_{SLI} and a sleeve outer diameter D_{SLO}, wherein the sleeve outer diameter D_{SLO} can be smaller than the balloon catheter inner diameter D_{BI}, to allow extension of the sleeve into the balloon catheter 210. In some examples, the sleeve 296 can comprise a rigid material that will prevent buckling or folding thereof, and will maintain constant values of its diameters D_{SLI} and D_{SLO} at all times.

The ultrasound sensor 260 defines an inner diameter that is smaller than the sleeve inner diameter D_{SLI}, such that the sensor 260 has a sensor proximal end surface 276 oriented proximally, facing the balloon catheter lumen 212 in the first position. Upon injection of inflation fluid via the balloon catheter 210 towards the balloon cavity 238, the injected fluid enters into the sleeve 296 and impinges against the sensor proximal end surface 276, thus applying a distally-oriented force striving to move the sensor 260 in the distal direction.

In some examples, a portion of the inflation fluid is also configured to flow past the sleeve 296 and the sensor 260 to fill the balloon cavity 238. For example, a gap may exist between the sensor distal portion 282 and the balloon wall 236 in the first position of the sensor 260, as illustrated in FIG. 13A, allowing inflation fluid to flow through this gap into the balloon cavity 238, thereby inflating the balloon 234 and optionally expanding the prosthetic valve 100.

As the balloon 234 is sufficiently inflated to a diameter greater than the outer diameter of the sensor D_{SO} and/or the sleeve D_{SLO}, the sensor 260 may be moved, due to the inflation fluid impinging against its proximal end surface 276, in the distal direction 20 to the second position, optionally until it contacts and is stopped by the stopper 252, as shown in FIG. 13B.

A total length of the sensor and sleeve L_{S} can be defined between the sensor distal portion 282 and the sleeve proximal end 298, which can be smaller, in some examples, than the length L_{BI} of the balloon intermediate section 242, so as to place the sleeve proximal end 298 distal to the inflation opening(s) 214 of the balloon catheter 210 in the second position, which will allow for continued undisturbed filling of the balloon cavity 238 by the inflation fluid, as well as suction of the inflation fluid when balloon deflation is required. It is to be understood that while a single sensor 260 is illustrated in FIGS. 13A-13B, the delivery apparatus 202^{d} can include a plurality of ultrasound sensors 260, which can be coupled to a carrier 284 and implemented in a manner similar to that described with respect to FIGS. 10A-10B, mutatis mutandis. When a plurality of sensors 260 are provided, the sleeve 296 will proximally extend from the proximal-most sensor 260, the sensor proximal end surface 276 against which the fluid impinges will be defined by the proximal-most sensor 260, and the total length L_{S} will be defined between the sensor distal portion 282 of the distal-most sensor 260 and the sleeve proximal end 298.

While not illustrated, it is to be understood that in some examples, a pull-wire, which can be similar to pull-wire 288 described with respect to delivery apparatus 202^{a}, can be attached to the sleeve 296 and extend proximally therefrom towards the handle 204, which can assist in repositioning the sensor 260 back towards the first position by pulling the pull-wire, and allow positioning of the sensor 260 proximal to the outflow end 102 of the valve 100 prior to crimping the valve, in a manner similar to that described with respect to FIG. 12, mutatis mutandis.

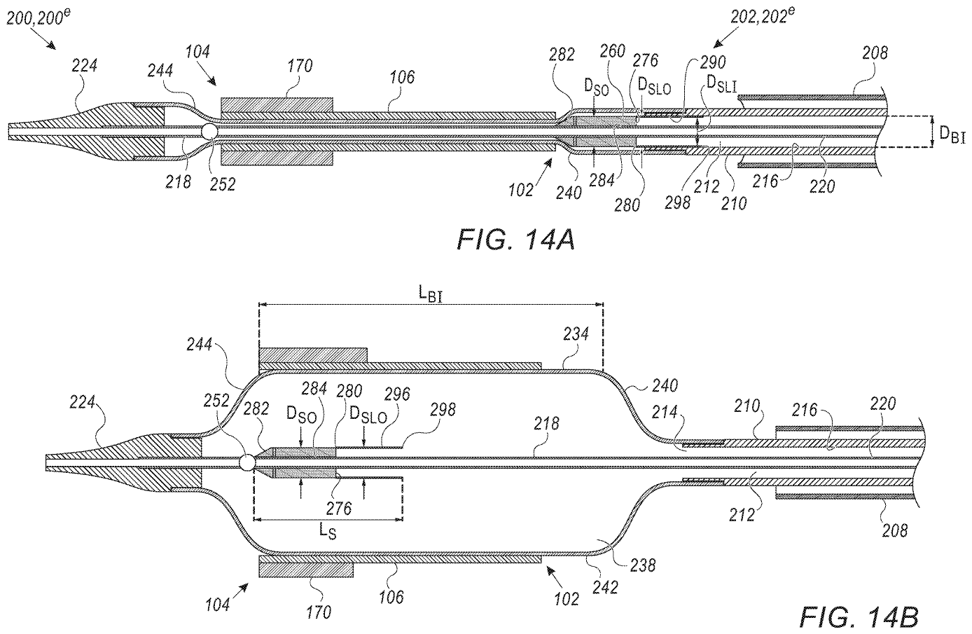

In some examples, the sensor distal portion 282 tapers in the distal direction to a smaller diameter. FIG. 14A is a cross-sectional view of a distal portion of an exemplary delivery assembly 200^{e} with the ultrasound sensor 260 shown in a first position while the balloon 234 is deflated and the prosthetic valve 100 is crimped thereover. FIG. 14B is a cross-sectional view of the delivery assembly 200^{e} with the balloon 234 inflated and the ultrasound sensor 260 moved to the second position. Delivery assembly 200^{e} is similar to any example described herein for delivery assembly 200^{d}, except that the sensor 260 of delivery apparatus 202^{e} comprises a tapering sensor distal portion 282, as illustrated in FIG. 14A. When a plurality of ultrasound sensors 260 are provided for delivery apparatus 202^{e}, at least the distal-most sensor 260 will have a tapering sensor distal portion 282.

In some examples, the sensor 260 can be wedged against the portion of balloon wall 236 covering the outflow end 102 of the crimped prosthetic valve 100 in the first position. When the sensor 260 is wedged in this manner, the distally-oriented force applied by inflation fluid injected through the balloon catheter 210 and impinging against the sensor proximal end surface 276 may be sufficient to facilitate initial opening of the frame 106 at the outflow end 102 as the arrow-shaped sensor distal portion 282 is pushed there-against, which can assist in initial valve opening and allow for distal advancement of the sensor 260 and filling of the balloon cavity 238 by the inflation fluid, in addition to, or in some examples, even in the absence of, a gap between the sensor 260 and the balloon wall 236.

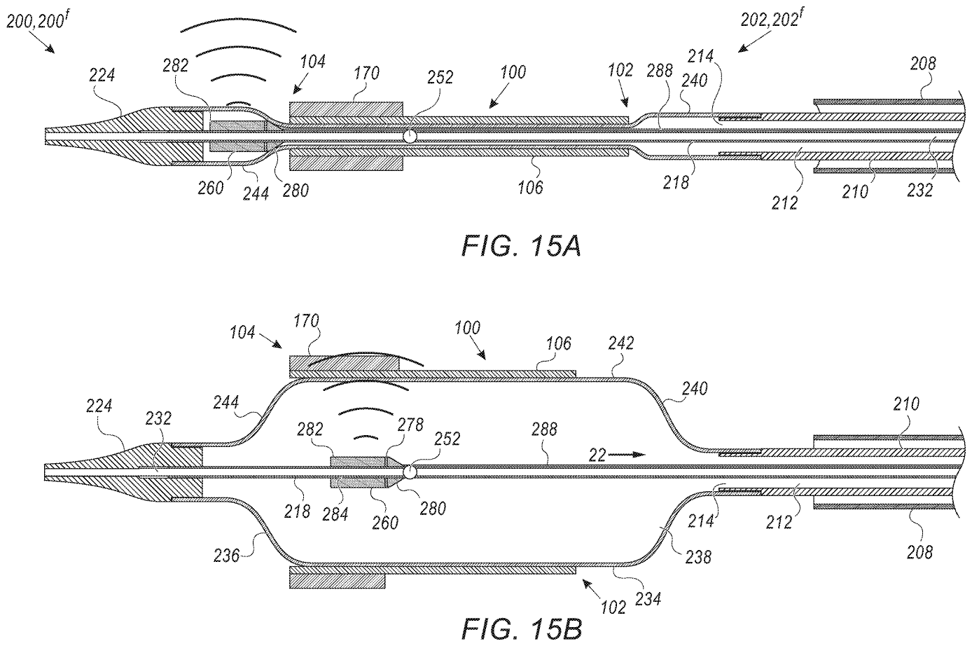

FIG. 15A is a cross-sectional view of a distal portion of an exemplary delivery assembly 200^{f} with the ultrasound sensor 260 shown in a first position while the balloon 234 is deflated and the prosthetic valve 100 is crimped thereover. FIG. 15B is a cross-sectional view of the delivery assembly 200^{f} with the balloon 234 inflated and the ultrasound sensor 260 moved to the second position. Delivery assembly 200^{f} is an exemplary implementation of delivery assembly 200, and thus can include any of the features described for delivery assembly 200 throughout the current disclosure, except that while delivery assembly 200 comprises a delivery apparatus 202 having one or more ultrasound sensor(s) 260 that can be actively or passively moved between the first and second positions according to various movement mechanisms, delivery assembly 200^{f} includes a delivery apparatus 202^{f} comprising an ultrasound sensor 260 configured to be proximally pulled from the first to the second position by a pull-wire 288 attached thereto.

In the example illustrated in FIG. 15A, the sensor 260 is shown to be positioned distal to the balloon intermediate section 242 in the first position, such as at the balloon distal section 244. In some examples, the sensor proximal portion 280 can be pressed against the portion of balloon wall 236 covering the inflow end 104 of the prosthetic valve 100, thus preventing proximally-oriented movement of the sensor 260 past the inflow end 104 as long as the prosthetic valve 100 remains crimped over the balloon intermediate section 242.

As mentioned, a pull-wire 288 can be attached to the ultrasound sensor 260, such as to the sensor proximal portion 280 and/or to a carrier 284 over which the sensor 260 can be disposed, extending proximally therethrough, such as thorough balloon catheter lumen 212, towards the handle 204. When the balloon 234 is inflated, such as to expand the prosthetic valve 100, the sensor 260 is no longer axially blocked, allowing the pull-wire 288 to be proximally pulled, pulling the sensor 260 therewith in the proximal direction 22 to the second position, as shown in FIG. 15B, optionally until the sensor proximal portion 280 engages with a stopper 252 at a desired axial position.

In some examples, the sensor proximal portion 280 can taper in the proximal direction to a narrower diameter. In this manner, the sensor proximal portion 280 can be wedged against the inflow end 104 of the prosthetic valve 100 in the first position, which can facilitate easier movement of the sensor 260 in the proximal direction 22 towards the second position, as the balloon 234 is inflated and the valve 100 is expanded. While the sensor 260 is shown to include a tapering proximal portion 280 in FIGS. 15A-15B, it is to be understood that this is merely optional, and that a sensor 260 of delivery apparatus 202^{f} can have any other shape along its proximal portion 280.

While not shown, it is to be understood that the pull-wire 288 can be configured to bias the sensor 260 in a proximal direction, such as by having the pull-wire 288 coupled to a spring which is loaded in the first position, so as to apply a pulling force, via pull-wire 288, on the sensor 260. It is to be understood that while a single sensor 260 is illustrated in FIGS. 15A-15B, the delivery apparatus 202^{f} can include a plurality of ultrasound sensors 260, which can be coupled to a carrier 284 and implemented in a manner similar to that described with respect to FIGS. 10A-10B, mutatis mutandis. While a pull-wire 288 is described with respect to delivery apparatus 202^{f}, this is not meant to be limiting, and it is to be understood that a pull-wire 288 can be similarly replaced by a rod configured to not only pull the sensor 260, but also configured to be pushed in a distal direction without buckling, thereby allowing it to axially move the sensor 260 in a distal direction 20, for example to reposition it to a first position prior to crimping the prosthetic valve 100 around the balloon 234.

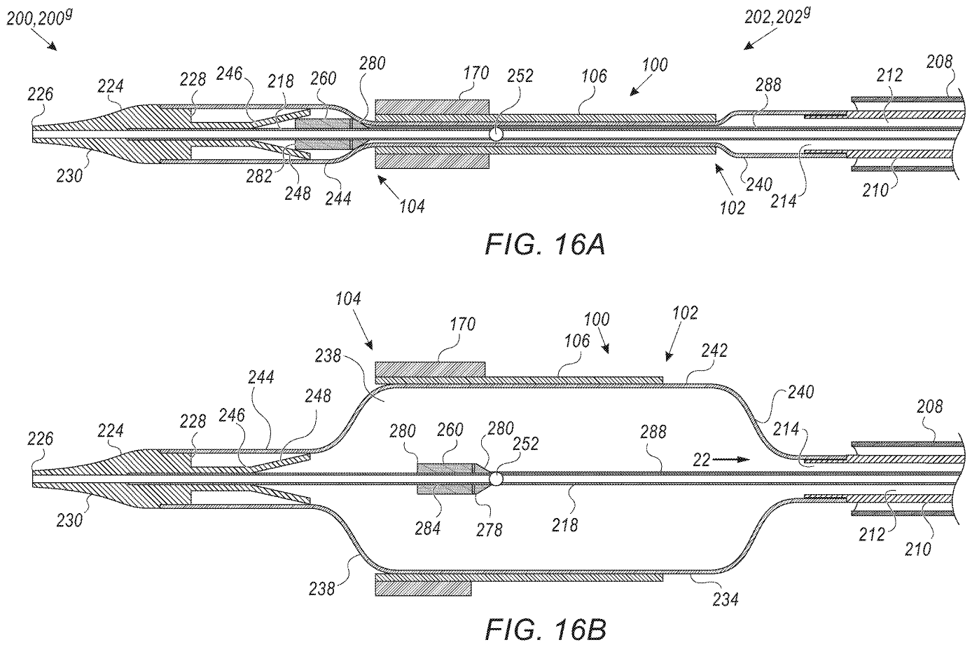

FIG. 16A is a cross-sectional view of a distal portion of an exemplary delivery assembly 200^{g} with the ultrasound sensor 260 shown in a first position while the balloon 234 is deflated and the prosthetic valve 100 is crimped thereover. FIG. 16B is a cross-sectional view of the delivery assembly 200^{g} with the balloon 234 inflated and the ultrasound sensor 260 moved to the second position. Delivery assembly 200^{g} is similar to any example described herein for delivery assembly 200^{e}, except that the sensor 260 of delivery apparatus 202^{g} is configured to reside inside a funnel-shaped portion of a proximal extension of the nosecone.

In some examples, the delivery apparatus 202 can further comprise a nosecone proximal extension 246 extending proximally from the nosecone 224, towards balloon distal section 244. The nosecone proximal extension 246 can have funnel-shaped portion 248 at its proximal end, which expands in diameter in the proximal direction, and may terminate at the border between the balloon distal section 244 and the balloon intermediate section 242. In such configurations, the sensor 260 can reside, at least partially, inside the extension funnel portion 248 in the first position, as illustrated in FIG. 16A. While not shown explicitly, it is to be understood that in some examples, the sensor distal portion 282 of a delivery apparatus 202^{g} can taper in the distal direction, as illustrated in FIGS. 14A-14B for example, which can wedge the sensor 260 inside the extension funnel portion 248 in the first position.

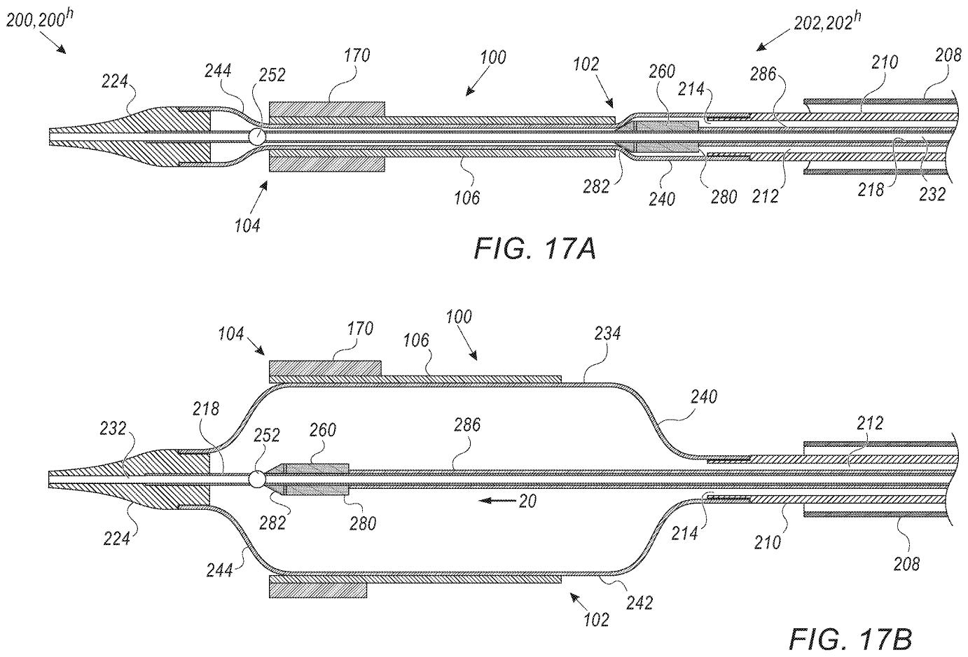

FIG. 17A is a cross-sectional view of a distal portion of an exemplary delivery assembly 200^{h} with the ultrasound sensor 260 shown in a first position while the balloon 234 is deflated and the prosthetic valve 100 is crimped thereover. FIG. 17B is a cross-sectional view of the delivery assembly 200^{h} with the balloon 234 inflated and the ultrasound sensor 260 moved to the second position. Delivery assembly 200^{h} is an exemplary implementation of delivery assembly 200, and thus can include any of the features described for delivery assembly 200 throughout the current disclosure, except that while delivery assembly 200 comprises a delivery apparatus 202 having one or more ultrasound sensor(s) 260 that can be actively or passively moved between the first and second positions according to various movement mechanisms, delivery assembly 200^{h} includes a delivery apparatus 202^{h} comprising an ultrasound sensor 260 attached to a sensor shaft 286 configured to slide inside the balloon catheter lumen 212, optionally around the nosecone shaft 218.

The sensor shaft 286 of delivery apparatus 202^{h} can be similar to the sensor shaft 286 of delivery apparatus 202^{c}, configured to be axially movable relative to the balloon catheter 210 and/or nosecone shaft 218, with the exception that the sensor shaft 286 of delivery apparatus 202^{h} is attached, at a distal end thereof, to the sensor 260, such as to sensor proximal portion 280, and/or to carrier 284 over which the sensor 260 can be disposed. In the example illustrated in FIG. 17A, the sensor 260 is shown to be situated in a first position which is proximal to the balloon intermediate section 242, optionally proximal to the outflow end 102 of the crimped prosthetic valve 100. Once the balloon begins to inflate and optionally expand the prosthetic valve 100, the sensor shaft 286 can be actively pushed in the distal direction 20, thereby pushing the sensor 260 therewith to the second position, optionally until the sensor is engaged with a stopper 252.

While not explicitly illustrated, it is to be understood that a delivery apparatus 202^{h} can be similarly implemented to include a sensor 260 positioned in a first position which is distal to the balloon intermediate section 242, optionally distal to the inflow end 104 of the crimped prosthetic valve 100, such as illustrated in FIG. 15A or 16A, and the sensor shaft 286 can be actively pulled in a proximal direction 22, thereby pulling the sensor 260 therewith to the second position, optionally until the sensor is engaged with a stopper 252, similar to the position illustrated, for example, in FIG. 15B or 16B.

While the distal portion 282 of the sensor 260 illustrated in FIGS. 17A-17B is shown to have a distally-tapering configuration, it is to be understood that this is shown by way of illustration and not limitation, and that any other shape of the sensor distal portion 282, as well as any other tapering or non-tapering shape of the sensor proximal portion 280, are contemplated. It is to be understood that while a single sensor 260 is illustrated in FIGS. 17A-17B, the delivery apparatus 202^{h} can include a plurality of ultrasound sensors 260, which can be coupled to a carrier 284 and implemented in a manner similar to that described with respect to FIGS. 10A-10B, mutatis mutandis.

While specific tapering or non-tapering shapes are illustrated for either the sensor proximal portion 280 and/or the sensor distal portion 282 throughout FIGS. 7A-7B, 10A-11B and 13A-13B, it is to be understood that any sensor 260 of any of the delivery assemblies 200^{a}, 200^{b}, 200^{c}, 200^{d}, 200^{e}, 200^{f}, 200^{g}, or 200^{h}, can have any desired shapes.

As mentioned above, the ultrasound sensor 260 can be utilized to acquire measurement signals when positioned in the second position, such as schematically illustrated in FIG. 15B, so as to provide an indication of the diameter of the balloon 234, the prosthetic valve 100, and/or the anatomy surrounding the balloon or prosthetic valve at the axial position of the sensor 260. In some examples, the ultrasound sensor 260 can be utilized to acquire measurement signals when positioned in the first position as well. For example, as schematically illustrated in FIG. 15A, measurement signals can be acquired by the ultrasound sensor 260 when positioned in the first position, distal to the balloon intermediate section 242 and/or prosthetic valve 100, optionally even prior to balloon inflation and/or valve expansion. This can assist in proper positioning of the balloon 234 and/or prosthetic valve 100 relative to a native annulus. For example, acquiring readings in this position can help identify whether the balloon distal section 244 is position at the level of the native annulus or inside the left ventricle, such as during aortic valve replacement procedures.

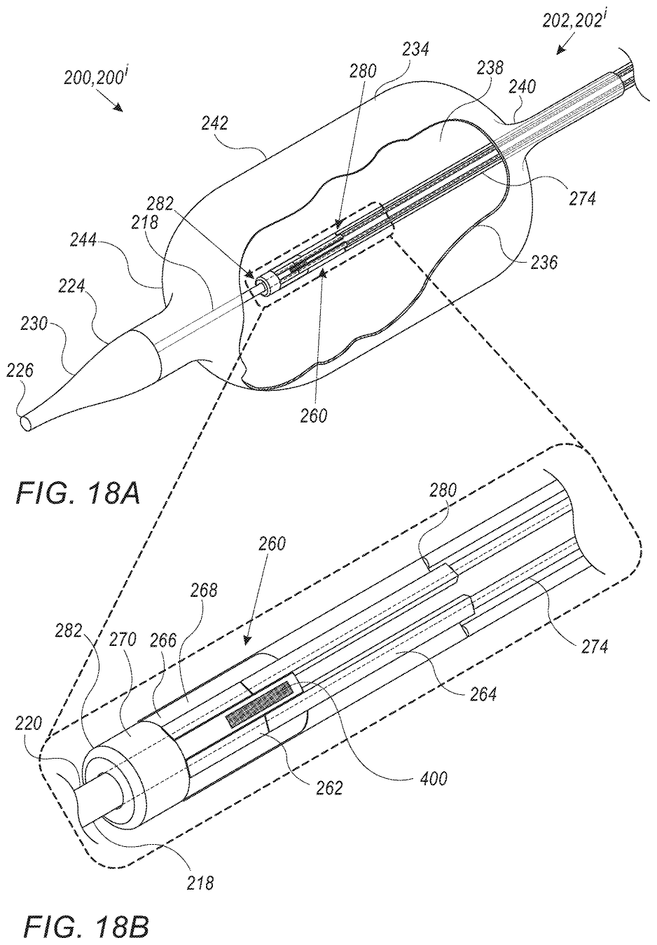

FIG. 18A shows a perspective cut-away view of a distal portion of an exemplary delivery apparatus 202ⁱ of an exemplary delivery assembly 200ⁱ.

Delivery assembly 200ⁱ is an exemplary implementation of delivery assembly 200, and thus can include any of the features described for delivery assembly 200 throughout the current disclosure, except that delivery apparatus 202ⁱ further comprises a temperature sensor 400. It is noted that although delivery assembly 200ⁱ is illustrated and described in relation to examples where only a single temperature sensor 400 is provided, this is not meant to be limiting in any way. In some examples (not shown), a plurality of temperature sensors 400 are provided.

FIG. 18B shows an enlarged perspective view of the ultrasound sensor 260 and the temperature sensor 400 of FIG. 18A.

In some examples, the temperature sensor 400 is secured to a respective spacer portion 268 of the separator 266. In some examples, the temperature sensor 400 is in communication with the sensor data unit 254 (not shown in FIGS. 18A-18B). In some examples, the communication between the temperature sensor 400 and the sensor data unit 254 is wireless. In some examples, the communication between the temperature sensor 400 and the sensor data unit 254 is via one or more wires (not shown).

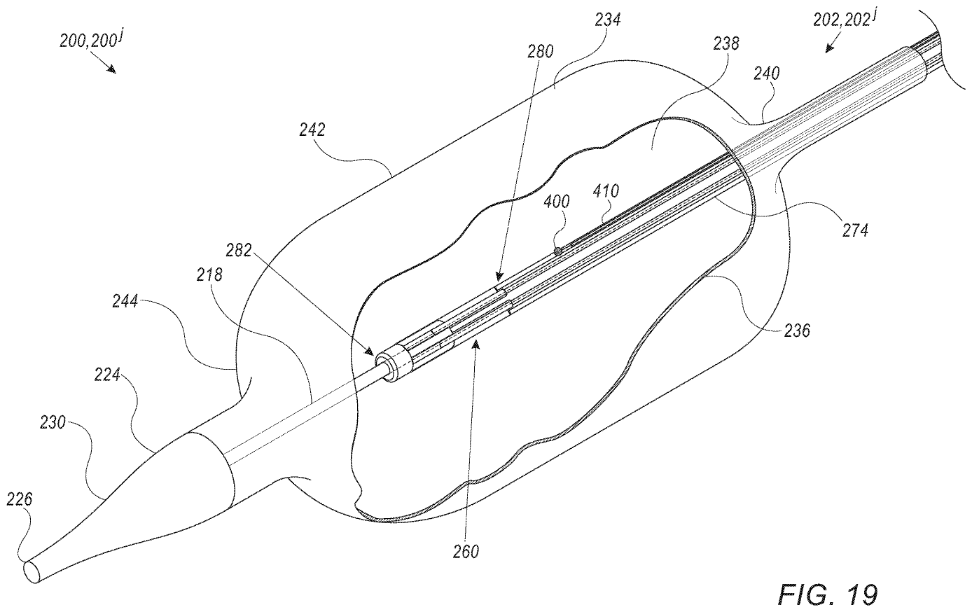

In some examples, as shown in FIG. 19, the temperature sensor 400 is secured to a dedicated sensor shaft 410 extending from the handle 204. In some examples, the sensor shaft 410 extends alongside the nosecone shaft 218. FIG. 19 shows a perspective cut-away view of a distal portion of an exemplary delivery apparatus 202^{j} of an exemplary delivery assembly 200^{j}. The temperature sensor 400 is illustrated in FIGS. 18A-18B as rectangular shaped, and illustrated in FIG. 19 as circular shaped, however this is not meant to be limiting in any way, and the temperature sensor 400 can be any suitable shape.

In some examples, the temperature sensor 400 can be any type of temperature sensor, including, but not limited to: an electrical temperature sensor, such as a thermistor, thermocouple, resistance thermometer or semiconductor-based temperature sensor; or an integrated circuit temperature sensor.

As described above, in some examples a diameter indication is calculated from the output of the ultrasound sensor 260. As known to those skilled in the art, the distance between the ultrasound sensor 260 and a respective object, such as the balloon wall 236 and/or the prosthetic valve 100 is based on the time it takes for the sound wave generated by the ultrasound sensor 260 to return thereto. The distance is then calculated as a multiplication of the time and the speed of sound. However, the speed of sound is a function of temperature. Therefore, the accuracy of the calculation of the diameter indication is dependent on the accuracy of the temperature value of the fluid within the balloon 234 that is used in the calculation.

In some examples, the sensor data unit 254 receives the output of the temperature sensor 400 and uses the temperature measurement of the temperature sensor 400 for determining the diameter indication. Since the temperature sensor 400 is positioned within the internal cavity 238 of the balloon 234, where the sound waves of the ultrasound sensor 260 are propagating, the calculation may be more accurate.

Although the above has been described in relation to examples where the temperature sensor 400 is positioned within the internal cavity 238 of the balloon 234, the temperature sensor 400 can be placed in other locations. These locations can be proximal to the balloon 234, at any point along the flow path of the inflation fluid of the balloon 234.

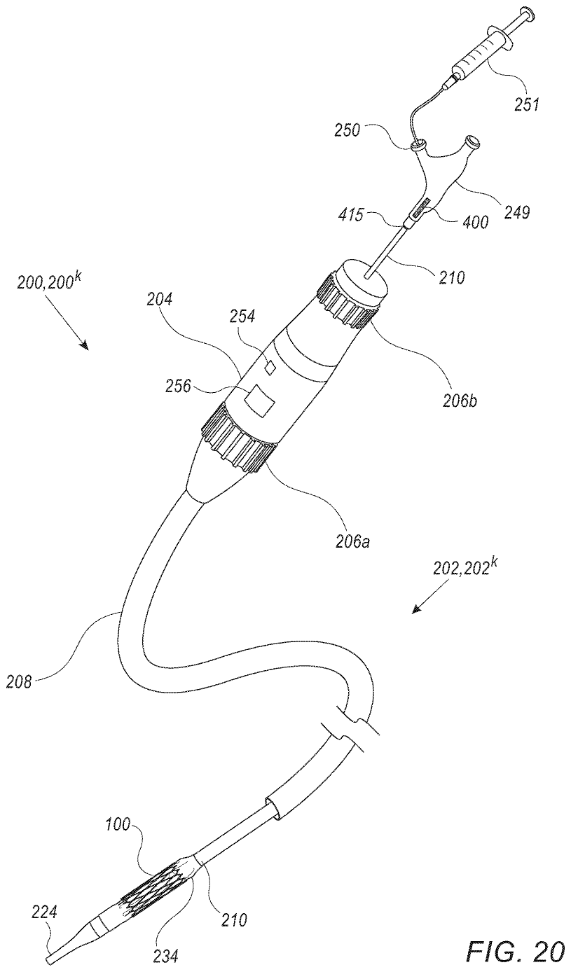

FIG. 20 shows a perspective view of a delivery apparatus 202^{k} of a delivery assembly 200^{k}. Delivery assembly 200^{k} is an exemplary implementation of delivery assembly 200, and thus can include any of the features described for delivery assembly 200 throughout the current disclosure, except that delivery apparatus 202^{k} comprises a temperature sensor 400 positioned within the proximal portion 249. As described above in relation to FIG. 2B, inflation fluid flows from the fluid source 251 through the fluid passageway 250 and into the balloon catheter 210.

In some examples, delivery assembly 200^{k} comprises a flow sensor 415. Although the flow sensor 415 is illustrated in FIG. 20 at the output of proximal portion 249, this is not meant to be limiting, and the flow sensor 415 can positioned anywhere within the fluid path of the inflation fluid. In some examples, the flow sensor 415 is in communication with the sensor data unit 254. In some examples, the communication is wireless. In some examples, the communication is via one or more wires (not shown). In some examples, the flow sensor 415 can be any type of flow sensor, including, but not limited to: a differential pressure flow sensor; or a thermal mass flow sensor.

In some examples, as described above, the sensor data unit 254 receives the output of the temperature sensor 400 and uses the temperature measurement of the temperature sensor 400 for determining the diameter indication. Since the temperature sensor 400 is positioned within the flow of the inflation fluid, the sensor data unit 254 can use the temperature value of the inflation fluid as a reference for determining the diameter indication.

Although the temperature of the fluid is measured by the temperature sensor 400, if the temperature sensor 400 is not positioned within the balloon 234, the measured temperature may be somewhat inaccurate since the temperature of the inflation fluid will increase as it flows through the body lumen.

In some examples, the measured temperature of the inflation fluid is adjusted by a predetermined function, and the adjusted temperature value is used for determining the diameter indication. In some examples, the predetermined function is determined based on previously performed tests where measurements are performed to determine the change in temperature when flowing from the fluid source 251 to the internal cavity 238 of the balloon 234.

In some examples, where a measured flow velocity of the inflation fluid is received from the flow sensor 415, the sensor data unit 254 further determines the estimated change in temperature based on the velocity value. For example, for a higher velocity, the change in temperature between the proximal portion 249 and the internal cavity 238 of the balloon 234 will be smaller since there is less time for the inflation fluid to heat up.

It is noted that the use of the temperature sensor 400 is not limited to the examples shown by delivery assemblies 200ⁱ, 200^{j} and 200^{k}, and the temperature sensor 400 can be used in any of delivery assemblies 200^{a}-200^{h}, or any other similar delivery assemblies.

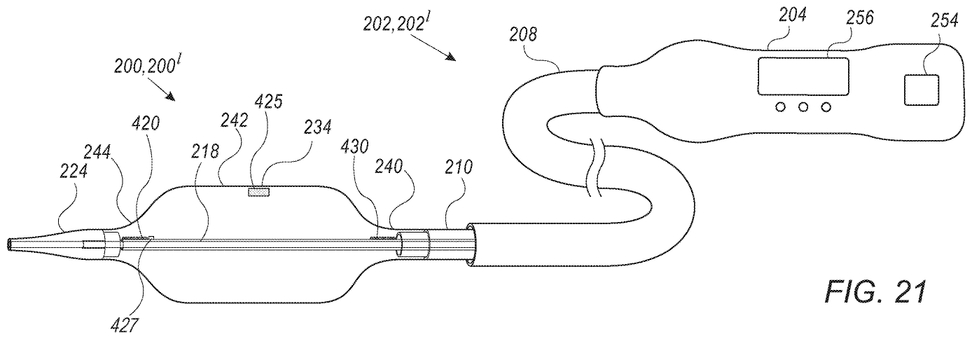

FIG. 21 shows a perspective view of a delivery apparatus 202^{l} of a delivery assembly 200^{l}. Delivery assembly 200^{l} is an exemplary implementation of delivery assembly 200, and thus can include any of the features described for delivery assembly 200 throughout the current disclosure, except that delivery apparatus 202^{l} comprises a pair of ultrasound transducers 420 and 430. Thus, in some examples, the ultrasound sensor is split into two parts-transducer 420 and transducer 430.

In some examples, both transducers 420 and 430 are secured to the nosecone shaft 218. In some examples, the transducer 420 is axially separated from the transducer 430. In some examples, the transducer 420 is positioned at the balloon distal section 244 of the balloon 234 and the transducer 430 is positioned at the balloon proximal section 240 of the balloon 234. In some examples, when a valve 100 is positioned over the balloon 234, the transducer 420 is positioned distal to the valve 100 and the transducer 430 is positioned proximal to the valve 100. Thus, in some examples, the transducers 420 and 430 don't affect the crimp profile of the valve 100.

In some examples, the delivery assembly apparatus 202^{l} further comprises a refractive surface 425 on the balloon 234. In some examples, the refractive surface 425 is positioned between the transducer 420 and the transducer 430. In some examples, the distance between the refractive surface 425 and the transducer 420 is substantially equal to the distance between the refractive surface 425 and the transducer 430. In some examples, the distance between the refractive surface 425 and the transducer 420 is different than the distance between the refractive surface 425 and the transducer 430.

In some examples, the delivery assembly apparatus 202^{l} further comprises a refractive element 427. In some examples, at least a portion of the refractive element 427 comprises a refractive surface. In some examples, the refractive element is positioned proximally to the transducer 420. In some examples, the refractive surface of the refractive element 427 blocks the line of sight between the transducer 420 and the transducer 430.

In some examples, as described above in relation to ultrasound sensor 260, the transducers 420 and 430 are in communication with the sensor data unit 254. The below will be described in relation to examples where the transducer 420 acts as a receiver and the transducer 430 acts as a transmitter, however this is not meant to limiting in any way. In some examples, the transducer 420 acts as a transmitter and the transducer 430 acts as a receiver.

In some examples, as described above, the sensor data unit 254 determines a diameter indication based on ultrasound signals. In some examples, the sensor data unit 254 controls the transducer 430 to generate ultrasound waves, and the sensor data unit 254 receives the output of the transducer 420. In some examples, each of the transducers 420 and 430 are angled such that at least a significant portion of ultrasound waves transmitted by the transducer 430 are received by the transducer 420.

In some examples, each of the transducers 420 and 430 are angled such that at least a significant portion of ultrasound waves transmitted by the transducer 430 arrive at the refractive surface 425. In some examples, the refractive surface 425 focuses the ultrasound waves towards the transducer 420.

In some examples, the refractive surface of the refractive element 427 blocks ultrasound waves propagating axially from the transducer 430 to the transducer 420. In some examples, this reduces the noise in the signal received at the transducer 420.

In some examples, the diameter indication is determined as described above, with the exception that the ultrasound waves travel is a generally triangular path, thereby the sensor data unit 254 calculates the height of such a triangle in order to determine the diameter indication.

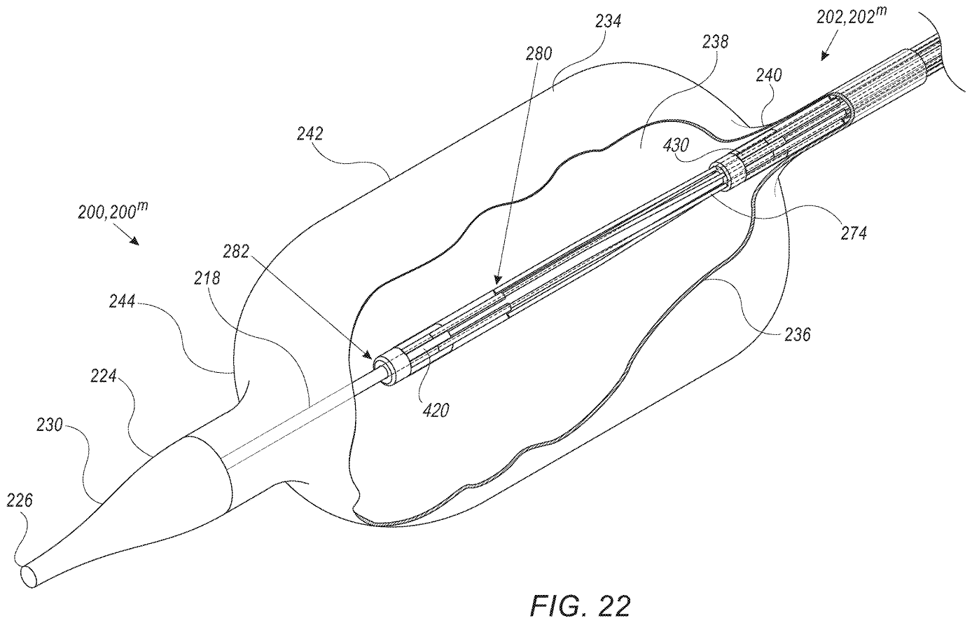

Although transducers 420 and 430 are illustrated in FIG. 21 at the proximal and distal sections of the balloon 234, this is not meant to be limiting. In some examples, transducers 420 and 430 are axially separated, but not necessarily at both ends. For example, FIG. 22 shows a perspective view of a delivery apparatus 202^{m} of a delivery assembly 200^{m}. Delivery assembly 200^{m} is an exemplary implementation of delivery assembly 200, and thus can include any of the features described for delivery assembly 200 throughout the current disclosure, except that delivery apparatus 202^{m} comprises a plurality of ultrasound transducers 430 positioned at the balloon proximal section 240 of the balloon 234 and a plurality of ultrasound transducers 420 positioned towards between the balloon proximal section 240 and the balloon distal section 244.

In some examples, ultrasound transducers 420 and 430 are positioned far enough towards the ends of the balloon 234 such that they are positioned distal and proximal, respectively, to a valve 100 when placed over the balloon 234, as described above.

In some examples, as described above, an ultrasound grating (not shown) is further provided and positioned between the ultrasound transducers 420 and the ultrasound transducers 430.

In some examples, as illustrated, the ultrasound transducers 420 and 430 are radially arrayed about the nosecone shaft 218, as described above in relation to the ultrasound transducers 262. As further described above, in some examples, the ultrasound transducers 420 and 430 are each in communication with the sensor data unit 254 via respective communication lines 274.

### Some Examples of the Disclosed Technology

Some examples of above-described technology are enumerated below. It should be noted that one feature of an example in isolation or more than one feature of the example taken in combination and, optionally, in combination with one or more features of one or more examples below are examples also falling within the disclosure of this application.

Example 1. A delivery assembly comprising: a delivery apparatus comprising: a balloon catheter defining a balloon catheter lumen; an inflatable balloon mounted on the balloon catheter and defining a balloon cavity that is in fluid communication with the balloon catheter lumen, the balloon comprising: a balloon proximal section attached to the balloon catheter; a balloon intermediate section extending distally from the balloon proximal section; and a balloon distal section extending distally from the balloon intermediate section; and at least one ultrasound sensor configured to move between a first position which is axially offset from the balloon intermediate section, and a second position which is axially aligned with the balloon intermediate section inside the balloon cavity.

Example 2. The delivery assembly of any example herein, particularly example 1, wherein the balloon is configured to transition between a deflated state and an inflated state, wherein the ultrasound sensor is configured to be in the first position in the deflated state of the balloon, and to be in the second position in the inflated state of the balloon.

Example 3. The delivery assembly of any example herein, particularly example 1 or 2, wherein the delivery apparatus further comprises a nosecone and a nosecone shaft extending proximally from the nosecone through the balloon cavity and through the balloon catheter lumen.

Example 4. The delivery assembly of any example herein, particularly example 3, wherein the at least one sensor is configured to axially slide over the nosecone shaft.

Example 5. The delivery assembly of any example herein, particularly any one of examples 1 to 4, wherein the at least one ultrasound sensor comprises a plurality of ultrasound sensors.

Example 6. The delivery assembly of any example herein, particularly example 5, wherein the plurality of ultrasound sensors are axially spaced from each other.

Example 7. The delivery assembly of any example herein, particularly example 6, wherein the plurality of ultrasound sensors are configured to simultaneously move between the first and second positions.

Example 8. The delivery assembly of any example herein, particularly any one of examples 3, wherein the delivery apparatus further comprises a spring configured to bias the at least one sensor to the second position.

Example 9. The delivery assembly of any example herein, particularly example 8, wherein the spring is attached, at a distal end of the spring, to the sensor.

Example 10. The delivery assembly of any example herein, particularly example 8, wherein the spring is attached, at a distal end of the spring, to a carrier over which the sensor is disposed, wherein the carrier is configured to axially slide along the nosecone shaft.

Example 11. The delivery assembly of any example herein, particularly any one of examples 9 or 10, wherein the spring is attached, at a proximal end of the spring, to the nosecone shaft.

Example 12. The delivery assembly of any example herein, particularly any one of examples 9 or 10, the spring is attached, at a proximal end of the spring, to a sensor shaft.

Example 13. The delivery assembly of any example herein, particularly example 12, wherein the at least one ultrasound sensor is axially spaced from the sensor shaft.

Example 14. The delivery assembly of any example herein, particularly any one of examples 12 or 13, wherein the sensor shaft is axially movable relative to the balloon catheter.

Example 15. The delivery assembly of any example herein, particularly any one of examples 12 to 14, wherein the sensor shaft is disposed around the nosecone shaft and is axially movable relative to the nosecone shaft.

Example 16. The delivery assembly of any example herein, particularly any one of examples 8 to 15, wherein the spring is configured to transition between a compressed configuration in the first position, and a free extended configuration in the second position.

Example 17. The delivery assembly of any example herein, particularly any one of examples 8 to 16, wherein the delivery apparatus further comprises a pull-wire extending proximally from the sensor.

Example 18. The delivery assembly of any example herein, particularly example 17, wherein the pull-wire extends through the balloon catheter lumen.

Example 19. The delivery assembly of any example herein, particularly any one of examples 17 or 18, wherein the pull-wire is configured, when proximally pulled, to compress the spring.

Example 20. The delivery assembly of any example herein, particularly any one of examples 17 to 19, wherein the pull-wire is configured, when proximally pulled, to proximally pull the at least one ultrasound sensor therewith.

Example 21. The delivery assembly of any example herein, particularly any one of examples 1 to 7, wherein the delivery apparatus further comprises a sleeve extending proximally from the at least one sensor.

Example 22. The delivery assembly of any example herein, particularly example 21, wherein the sleeve comprises a rigid material.

Example 23. The delivery assembly of any example herein, particularly any one of examples 21 or 22, wherein an outer diameter defined by the sleeve is smaller than an inner diameter defined by the balloon catheter.

Example 24. The delivery assembly of any example herein, particularly any one of examples 21 to 23, the at least one ultrasound sensor defines a sensor proximal end surface, and wherein, when inflation fluid is injected into the balloon catheter lumen, the injection fluid is configured to impinge against the sensor proximal end surface, when in the first position, so as to distally bias the at least one ultrasound sensor towards the second position.

Example 25. The delivery assembly of any example herein, particularly any one of examples 21 to 24, wherein a length defined between a distal portion of the at least one ultrasound sensor and a proximal end of the sleeve is less than a length defined by the balloon intermediate section.

Example 26. The delivery assembly of any example herein, particularly example 3, wherein the at least one sensor is distal to the balloon intermediate section in the first position.

Example 27. The delivery assembly of any example herein, particularly example 26, wherein the delivery apparatus further comprises a pull-wire extending proximally from the sensor.

Example 28. The delivery assembly of any example herein, particularly example 27, wherein the pull-wire extends through the balloon catheter lumen.

Example 29. The delivery assembly of any example herein, particularly any one of examples 27 or 28, wherein the pull-wire is configured, when proximally pulled, to proximally pull the at least one ultrasound sensor therewith.

Example 30. The delivery assembly of any example herein, particularly any one of examples 26 to 29, wherein the delivery apparatus further comprises a nosecone proximal extension extending proximally from the nosecone, the nosecone proximal extension disposed around the nosecone shaft and comprising an extension funnel portion at a proximal end thereof.

Example 31. The delivery assembly of any example herein, particularly example 30, wherein at least part of the at least one ultrasound sensor is configured to reside inside the extension funnel portion in the first position.

Example 32. The delivery assembly of any example herein, particularly any one of examples 30 or 31, wherein the extension funnel portion is distal to the balloon intermediate section.

Example 33. The delivery assembly of any example herein, particularly any one of examples 1 to 7, wherein the delivery apparatus further comprises a sensor shaft extending proximally from the sensor.

Example 34. The delivery assembly of any example herein, particularly example 33, wherein the sensor shaft extends through the balloon catheter lumen.

Example 35. The delivery assembly of any example herein, particularly any one of examples 33 or 34, wherein the sensor shaft is configured, when axially moved, to axially move the at least one ultrasound sensor therewith.

Example 36. The delivery assembly of any example herein, particularly example 3, wherein the delivery apparatus further comprises a stopper disposed around the nosecone shaft inside the balloon cavity.

Example 37. The delivery assembly of any example herein, particularly example 36, wherein the stopper is axially positioned between the balloon proximal section and the balloon distal section.

Example 38. The delivery assembly of any example herein, particularly any one of examples 36 or 37, wherein the at least one ultrasound sensor is configured to engage with the stopper when moved from the first to the second position, and is prevented from further axial movement in a direction from the first towards the second position by the stopper.

Example 39. The delivery assembly of any example herein, particularly any one of examples 36 to 38, wherein the stopper is spherical.

Example 40. The delivery assembly of any example herein, particularly any one of examples 36 to 38, wherein the stopper is wedge-shaped.

Example 41. The delivery assembly of any example herein, particularly any one of examples 1 to 40, wherein the at least one ultrasound sensor comprises at least one radiopaque marker.

Example 42. The delivery assembly of any example herein, particularly any one of examples 1 to 41, wherein the at least one sensor comprises a tapering sensor proximal portion.

Example 43. The delivery assembly of any example herein, particularly any one of examples 1 to 42, wherein the at least one sensor comprises a tapering sensor distal portion.

Example 44. The delivery assembly of any example herein, particularly any one of examples 1 to 41, further comprising a prosthetic valve disposed around the balloon intermediate section, the prosthetic valve extending between an inflow end and an outflow end and comprising a frame movable between a crimped state and an expanded state.

Example 45. The delivery assembly of any example herein, particularly example 44, wherein the prosthetic valve, when in the crimped state, is axially aligned with the at least one ultrasound sensor in the first position, and wherein the prosthetic valve, when in the expanded state, is axially aligned with the at least one ultrasound sensor in the second position.

Example 46. The delivery assembly of any example herein, particularly any one of examples 44 or 45, wherein the at least one ultrasound sensor is proximal to the outflow end of the prosthetic valve in the first position.

Example 47. The delivery assembly of any example herein, particularly example 46, wherein, when in the crimped state, the outflow end of the prosthetic valve prevents the at least one ultrasound sensor from axially moving distally towards the second position.

Example 48. The delivery assembly of any example herein, particularly any one of examples 46 or 47, wherein the at least one sensor comprises a tapering sensor distal portion configured to wedge against the outflow end of the prosthetic valve in the first position.

Example 49. The delivery assembly of any example herein, particularly any one of examples 44 or 45, wherein the at least one ultrasound sensor is distal to the outflow end of the prosthetic valve in the first position.

Example 50. The delivery assembly of any example herein, particularly example 49, wherein, when in the crimped state, the inflow end of the prosthetic valve prevents the at least one ultrasound sensor from axially moving proximally towards the second position.

Example 51. The delivery assembly of any example herein, particularly any one of examples 49 or 50, wherein the at least one sensor comprises a tapering sensor proximal portion configured to wedge against the inflow end of the prosthetic valve in the first position.

Example 52. The delivery assembly of any example herein, particularly any one of examples 44 to 51, wherein the at least one ultrasound sensor is closer to the inflow end of the prosthetic valve than to the outflow end, in the second position of the at least one ultrasound sensor.

Example 53. The delivery assembly of any example herein, particularly any one of examples 1 to 52, wherein an inner diameter defined by the balloon catheter is greater than an outer diameter defined by the at least one ultrasound sensor.

Example 54. The delivery assembly of any example herein, particularly any one of examples 1 to 43, wherein the delivery apparatus further comprises a sensor data unit in communication with the at least one ultrasound sensor.

Example 55. The delivery assembly of any example herein, particularly example 54, wherein the sensor data unit is configured, based at least in part on an output of the at least one ultrasound sensor, to determine a diameter indication of the balloon.

Example 56. The delivery assembly of any example herein, particularly example 54, wherein the sensor data unit is configured, based at least in part on an output of the at least one ultrasound sensor, to determine a diameter indication of an anatomical wall around the balloon.

Example 57. The delivery assembly of any example herein, particularly example 54, further comprising a prosthetic valve disposed around the balloon intermediate section, wherein the sensor data unit is configured, responsive to an output of the at least one ultrasound sensor, to determine a diameter indication of the prosthetic valve.

Example 58. The delivery assembly of any example herein, particularly any one of examples 1 to 57, wherein the at least one ultrasound sensor comprises a plurality of ultrasound transducers.

Example 59. The delivery assembly of any example herein, particularly example 58, wherein each of the plurality of ultrasound transducers is directed towards a balloon wall of the balloon in the second position of the at least one ultrasound sensor.

Example 60. The delivery assembly of any example herein, particularly any one of examples 58 or 59, wherein the plurality of ultrasound transducers comprises at least two ultrasound transducers which are diametrically opposite to each other.

Example 61. The delivery assembly of any example herein, particularly any one of examples 58 to 60, wherein the at least one sensor further comprises a separator, and wherein the plurality of ultrasound transducers are attached to the separator.

Example 62. The delivery assembly of any example herein, particularly example 61, wherein the separator comprises a non-conductive material.

Example 63. The delivery assembly of any example herein, particularly any one of examples 61 or 62, wherein the separator comprises a plurality of depressions, and wherein the plurality of ultrasound transducers are situated inside the plurality of depressions.

Example 64. The delivery assembly of any example herein, particularly any one of examples 61 to 63, wherein the plurality of ultrasound transducers are circumferentially spaced from each other by spacer portions of the separator.

Example 65. A method comprising: advancing a delivery assembly that comprises a delivery apparatus comprising a balloon mounted on a balloon catheter and at least one ultrasound sensor, to a target site of treatment, while the at least one ultrasound sensor is in a first position which is axially offset from an intermediate balloon section of the balloon; inflating the balloon by streaming inflation fluid into a cavity of the balloon; and axially moving the at least one ultrasound sensor to a second position which is aligned with the intermediate balloon section inside the balloon cavity.

Example 66. The method of any example herein, particularly example 65, wherein the inflating the balloon and the axially moving the at least one ultrasound sensor are performed simultaneously.

Example 67. The method of any example herein, particularly example 65, wherein the axially moving the at least one ultrasound sensor is performed during the inflating the balloon.

Example 68. The method of any example herein, particularly any one of examples 65 to 67, wherein the axially moving the at least one ultrasound sensor comprises sliding the at least one ultrasound sensor over a nosecone shaft extending through the balloon cavity.

Example 69. The method of any example herein, particularly any one of examples 65 to 68, wherein the first position of the at least one ultrasound sensor is proximal to the intermediate balloon section.

Example 70. The method of any example herein, particularly example 69, wherein the advancing the delivery assembly comprises maintaining a spring attached to the at least one ultrasound sensor in a compressed loaded state configured to distally bias the at least one ultrasound sensor towards the second position.

Example 71. The method of any example herein, particularly example 70, wherein the axially moving the at least one ultrasound sensor comprises allowing the spring to move to a free extended state thereof.

Example 72. The method of any example herein, particularly example 69, wherein the axially moving the at least one ultrasound sensor comprises injecting the inflation fluid into a sleeve extending proximally from the at least one ultrasound sensor, such that the inflation fluid impinges against a proximal surface of the at least one ultrasound sensor and biases the at least one ultrasound sensor distally towards the second position.

Example 73. The method of any example herein, particularly example 72, wherein the sleeve comprises a rigid material.

Example 74. The method of any example herein, particularly example 72 or 73, wherein, in the second position, a proximal end of the sleeve is distal to an inflation opening of the balloon catheter.

Example 75. The method of any example herein, particularly example 69, wherein the axially moving the at least one ultrasound sensor comprises distally pushing a sensor shaft attached to the at least one ultrasound sensor.

Example 76. The method of any example herein, particularly example 75, wherein the sensor shaft extends proximally from the at least one ultrasound sensor and through the balloon catheter.

Example 77. The method of any example herein, particularly any one of examples 75 or 76, wherein the sensor shaft extends around a nosecone shaft attached to a nosecone of the delivery apparatus.

Example 78. The method of any example herein, particularly any one of examples 69 to 77, wherein the delivery assembly further comprises a prosthetic valve, wherein the advancing the delivery assembly comprises maintaining the prosthetic valve in a crimped state around the balloon intermediate section such that an outflow end of the prosthetic valve prevents the at least one ultrasound sensor from moving distally towards the second position.

Example 79. The method of any example herein, particularly example 78, wherein the inflating the balloon further comprises expanding the prosthetic valve to a diameter to no longer prevents the at least one ultrasound sensor from moving distally towards the second position.

Example 80. The method of any example herein, particularly any one of examples 78 or 79, wherein the at least one ultrasound sensor further comprises a distally tapering sensor distal portion, and wherein the advancing the delivery assembly further comprises wedging the sensor distal portion against the outflow end of the prosthetic valve.

Example 81. The method of any example herein, particularly any one of examples 65 to 68, wherein the first position of the at least one ultrasound sensor is distal to the intermediate balloon section.

Example 82. The method of any example herein, particularly example 81, wherein the axially moving the at least one ultrasound sensor comprises proximally pulling a pull-wire attached to the at least one ultrasound sensor.

Example 83. The method of any example herein, particularly example 82, wherein the pull-wire extends proximally from the at least one ultrasound sensor and through the balloon catheter.

Example 84. The method of any example herein, particularly example 81, wherein the axially moving the at least one ultrasound sensor comprises proximally pulling a sensor shaft attached to the at least one ultrasound sensor.

Example 85. The method of any example herein, particularly example 84, wherein the sensor shaft extends proximally from the at least one ultrasound sensor and through the balloon catheter.

Example 86. The method of any example herein, particularly any one of examples 81 to 85, wherein the delivery assembly further comprises a prosthetic valve, wherein the advancing the delivery assembly comprises maintaining the prosthetic valve in a crimped state around the balloon intermediate section such that an inflow end of the prosthetic valve prevents the at least one ultrasound sensor from moving proximally towards the second position.

Example 87. The method of any example herein, particularly example 86, wherein the inflating the balloon further comprises expanding the prosthetic valve to a diameter that no longer prevents the at least one ultrasound sensor from moving proximally towards the second position.

Example 88. The method of any example herein, particularly any one of examples 86 or 87, wherein the at least one ultrasound sensor further comprises a proximally tapering sensor proximal portion, and wherein the advancing the delivery assembly further comprises wedging the sensor proximal portion against the inflow end of the prosthetic valve.

Example 89. The method of any example herein, particularly any one of examples 65 to 88, wherein the axially moving the at least one ultrasound sensor comprises engaging the at least one ultrasound sensor with a stopper that prevents further axial movement of the at least one ultrasound sensor in a direction from the first to the second position.

Example 90. The method of any example herein, particularly any one of examples 65 to 89, further comprising, after the axially moving the at least one ultrasound sensor, measuring, by at least one ultrasound transducer of the ultrasound sensor, a radial distance to the balloon or another rigid structure surround the balloon.

Example 91. The method of any example herein, particularly any one of examples 65 to 89, further comprising, before the axially moving the at least one ultrasound sensor, measuring, by at least one ultrasound transducer of the ultrasound sensor, a radial distance to the balloon or another rigid structure surround the balloon.

Example 92. A delivery assembly comprising: a delivery apparatus comprising: a balloon catheter defining a balloon catheter lumen; an inflatable balloon mounted on the balloon catheter and defining a balloon cavity that is in fluid communication with the balloon catheter lumen; at least one ultrasound sensor; and a temperature sensor.

Example 93. The delivery assembly of any example herein, particularly example 92, wherein the delivery apparatus further comprises a sensor data unit in communication with the at least one ultrasound sensor.

Example 94. The delivery assembly of any example herein, particularly example 93, wherein the sensor data unit is configured, based at least in part on an output of the at least one ultrasound sensor, to determine a diameter indication of the balloon.

Example 95. The delivery assembly of any example herein, particularly example 94, wherein the determination of the diameter indication of the balloon is further based on the output of the temperature sensor.

Example 96. The delivery assembly of any example herein, particularly example 95, wherein the delivery apparatus further comprises a flow sensor, the determination of the diameter indication of the balloon further based on the output of the flow sensor.

Example 97. The delivery assembly of any example herein, particularly example 96, wherein the sensor data unit adjusts a temperature value output by the temperature sensor based at least in part on the output of the flow sensor, the determination of the diameter indication of the balloon based at least in part on the adjusted temperature value.

Example 98. The delivery assembly of any example herein, particularly example 93, wherein the sensor data unit is configured, based at least in part on an output of the at least one ultrasound sensor, to determine a diameter indication of an anatomical wall around the balloon.

Example 99. The delivery assembly of any example herein, particularly example 98, wherein the determined diameter indication of the anatomical wall around the balloon is further based on the output of the temperature sensor.

Example 100. The delivery assembly of any example herein, particularly example 99, wherein the delivery apparatus further comprises a flow sensor, the determination of the diameter indication of the anatomical wall further based on the output of the flow sensor.

Example 101. The delivery assembly of any example herein, particularly example 100, wherein the sensor data unit adjusts a temperature value output by the temperature sensor based at least in part on the output of the flow sensor, the determination of the diameter indication of the anatomical wall based at least in part on the adjusted temperature value.

Example 102. The delivery assembly of any example herein, particularly example 93, further comprising a prosthetic valve disposed around the balloon, wherein the sensor data unit is configured, responsive to an output of the at least one ultrasound sensor, to determine a diameter indication of the prosthetic valve.

Example 103. The delivery assembly of any example herein, particularly example 102, wherein the determination of the diameter indication of the prosthetic valve is further based on the output of the temperature sensor.

Example 104. The delivery assembly of any example herein, particularly example 103, wherein the delivery apparatus further comprises a flow sensor, the determination of the diameter indication of the prosthetic valve further based on the output of the flow sensor.

Example 105. The delivery assembly of any example herein, particularly example 104 wherein the sensor data unit adjusts a temperature value output by the temperature sensor based at least in part on the output of the flow sensor, the determination of the diameter indication of the prosthetic valve based at least in part on the adjusted temperature value.

Example 106. The delivery assembly of any example herein, particularly any one of examples 92 to 105, wherein the temperature sensor is positioned within the balloon cavity.

Example 107. The delivery assembly of any example herein, particularly any one of examples 92 to 105, wherein the delivery apparatus comprises a handle, the balloon catheter extending distally from the handle, and wherein the temperature sensor is positioned proximal to the handle.

Example 108. The delivery assembly of any example herein, particularly any one of examples 92 to 107, wherein the at least one ultrasound sensor comprises a plurality of ultrasound transducers.

Example 109. The delivery assembly of any example herein, particularly example 108, wherein each of the plurality of ultrasound transducers is directed towards a balloon wall of the balloon.

Example 110. The delivery assembly of any example herein, particularly any one of examples 108 or 109, wherein the plurality of ultrasound transducers comprises at least two ultrasound transducers which are diametrically opposite to each other.

Example 111. The delivery assembly of any example herein, particularly any one of examples 108 to 110, wherein the at least one sensor further comprises a separator, and wherein the plurality of ultrasound transducers are attached to the separator.

Example 112. The delivery assembly of any example herein, particularly example 111, wherein the separator comprises a non-conductive material.

Example 113. The delivery assembly of any example herein, particularly any one of examples 111 or 112, wherein the separator comprises a plurality of depressions, and wherein the plurality of ultrasound transducers are situated inside the plurality of depressions.

Example 114. The delivery assembly of any example herein, particularly any one of examples 111 to 113, wherein the plurality of ultrasound transducers are circumferentially spaced from each other by spacer portions of the separator.

Example 115. The delivery assembly of any example herein, particularly any one of examples 111 to 114, wherein the temperature sensor is attached to the separator.

Example 116. The delivery assembly of any example herein, particularly any one of examples 111 to 115, wherein the delivery apparatus further comprises a nosecone and a nosecone shaft extending proximally from the nosecone through the balloon cavity and through the balloon catheter lumen, and wherein the separator is attached to the nosecone shaft.

Example 117. The delivery assembly of any example herein, particularly any example herein, particularly any one of examples 92 to 110, wherein the delivery apparatus further comprises a nosecone and a nosecone shaft extending proximally from the nosecone through the balloon cavity and through the balloon catheter lumen, and wherein the temperature sensor is secured to the nosecone shaft.

Example 118. The delivery assembly of any example herein, particularly any one of examples 92 to 110, wherein the delivery apparatus further comprises: a nosecone and a nosecone shaft extending proximally from the nosecone through the balloon cavity and through the balloon catheter lumen; and a sensor shaft extending alongside the nosecone shaft, and wherein the temperature sensor is attached to the sensor shaft.

Example 119. A method comprising: advancing a delivery assembly that comprises a delivery apparatus comprising a balloon mounted on a balloon catheter and at least one ultrasound sensor, to a target site of treatment; inflating the balloon by streaming inflation fluid into a cavity of the balloon; measuring a temperature of the inflation fluid; and based at least in part on an output of the at least one ultrasound sensor and the measured temperature of the inflation fluid, determining a diameter indication, wherein the determined diameter indication is of one or more of: the balloon; an anatomical wall around the balloon; or a prosthetic valve disposed around the balloon.

Example 120. The method of any example herein, particularly example 119, further comprising: measuring a flow velocity of the inflation fluid; and adjusting the measured temperature based at least in part on the measured flow velocity, the determination of the diameter indication based at least in part on the adjusted temperature.

Example 121. The method of any example herein, particularly any one of examples 119 or 120, wherein the delivery apparatus comprises a temperature sensor positioned within the cavity of the balloon, wherein the temperature is measured by the temperature sensor.

Example 122. The method of any example herein, particularly any one of examples 119 or 120, wherein the delivery apparatus comprises: a handle, the balloon catheter extending distally from the handle; and a temperature sensor positioned proximal to the handle, and wherein the temperature is measured by the temperature sensor.

Example 123. The method of any example herein, particularly any one of examples 119 to 122, wherein the at least one ultrasound sensor comprises a plurality of ultrasound transducers.

Example 124. The method of any example herein, particularly example 123, wherein each of the plurality of ultrasound transducers is directed towards a balloon wall of the balloon.

Example 125. A delivery assembly comprising: a delivery apparatus comprising: a balloon catheter defining a balloon catheter lumen; an inflatable balloon mounted on the balloon catheter and defining a balloon cavity that is in fluid communication with the balloon catheter lumen; at least one first ultrasound transducer; and at least one second ultrasound transducer axially offset from the at least one first ultrasound transducer.

Example 126. The delivery assembly of any example herein, particularly example 125, wherein the delivery apparatus further comprises a sensor data unit in communication with the at least one first ultrasound transducer and the at least one second ultrasound transducer.

Example 127. The delivery assembly of any example herein, particularly example 126, wherein the sensor data unit is configured to: control the at least one first ultrasound transducer to generate ultrasound waves; and detect the ultrasound waves at the at least one second ultrasound transducer.

Example 128. The delivery assembly of any example herein, particularly example 126, wherein the sensor data unit is configured, based at least in part on the detected ultrasound waves, to determine a diameter indication of the balloon.

Example 129. The delivery assembly of any example herein, particularly example 126, wherein the sensor data unit is configured, based at least in part on the detected ultrasound waves, to determine a diameter indication of an anatomical wall around the balloon.

Example 130. The delivery assembly of any example herein, particularly example 126, further comprising a prosthetic valve disposed around the balloon, wherein the sensor data unit is configured, based at least in part on the detected ultrasound waves, to determine a diameter indication of the prosthetic valve.

Example 131. The delivery assembly of any example herein, particularly example 125, wherein a portion of the balloon comprises a refractive surface, the refractive surface of the balloon positioned between the at least one first ultrasound transducer and the at least one second ultrasound transducer.

Example 132. The delivery assembly of any example herein, particularly example 131, wherein the at least one first ultrasound transducer is directed towards the refractive surface of the balloon.

Example 133. The delivery assembly of any example herein, particularly any one of examples 131 or 132, wherein the at least one second ultrasound transducer is directed towards the refractive surface of the balloon.

Example 134. The delivery assembly of any example herein, particularly any one of examples 125 to 133, further comprising a refractive element comprising a refractive surface positioned between the at least one first ultrasound transducer and the at least one second ultrasound transducer such that a line of sight between the at least one first ultrasound transducer and the at least one second ultrasound transducer is blocked.

Example 135. The delivery assembly of any example herein, particularly any one of examples 125 to 134, wherein the at least one first ultrasound transducer comprises a plurality of first ultrasound transducers and the at least one second ultrasound transducer comprises a plurality of second ultrasound transducers.

Example 136. The delivery assembly of any example herein, particularly example 135, wherein the plurality of ultrasound transducers comprises at least two ultrasound transducers which are diametrically opposite to each other.

Example 137. The delivery assembly of any example herein, particularly any one of examples 135 to 136, further comprising a pair of separators, wherein the plurality of first ultrasound transducers are attached to a first of the pair of separators and the plurality of second ultrasound transducers are attached to a second of the pair of separators.

Example 138. The delivery assembly of any example herein, particularly example 137, wherein each of the first separator and the second separator comprises a non-conductive material.

Example 139. The delivery assembly of any example herein, particularly any one of examples 137 or 138, wherein each of the first separator and the second separator comprises a plurality of depressions, and wherein the plurality of first ultrasound transducers are situated inside the plurality of depressions of the first separator and the plurality of second ultrasound transducers are situated inside the plurality of depressions of the second separator.

Example 140. The delivery assembly of any example herein, particularly any one of examples 137 to 139, wherein the plurality of first ultrasound transducers are circumferentially spaced from each other by spacer portions of the first separator and the plurality of second ultrasound transducers are circumferentially spaced from each other by spacer portions of the second separator.

Example 141. The delivery assembly of any example herein, particularly any one of examples 137 to 140, wherein the delivery apparatus further comprises a nosecone and a nosecone shaft extending proximally from the nosecone through the balloon cavity and through the balloon catheter lumen, and wherein the first separator and the second separator are attached to the nosecone shaft.

Example 142. A method comprising: advancing a delivery assembly to a target site of treatment, wherein the delivery assembly comprises a delivery apparatus comprising a balloon mounted on a balloon catheter, at least one first ultrasound transducer and at least one second ultrasound transducer axially offset from the at least one first ultrasound transducer; inflating the balloon by streaming inflation fluid into a cavity of the balloon; measuring a temperature of the inflation fluid; generating ultrasound waves using the at least one first ultrasound transducer; and detecting the ultrasound waves using the at least one second ultrasound transducer.

Example 143. The method of any example herein, particularly example 142, further comprising, based at least in part on the detected ultrasound waves, determining a diameter indication of the balloon.

Example 144. The method of any example herein, particularly example 142, further comprising, based at least in part on the detected ultrasound waves, determining a diameter indication of an anatomical wall around the balloon.

Example 145. The method of any example herein, particularly example 142, further comprising, based at least in part on detected ultrasound waves, determining a diameter indication of a prosthetic valve disposed around the balloon.

Example 146. The method of any example herein, particularly any one of examples 142 to 145, further comprising directing the generated ultrasound waves to a refractive surface of the balloon, the refractive surface of the balloon positioned between the at least one first ultrasound transducer and the at least one second ultrasound transducer.

Example 147. The method of any example herein, particularly example 146, further comprising directing the at least one second ultrasound transducer to detect the ultrasound waves from the refractive surface of the balloon.

Example 148. The method of any example herein, particularly any one of examples 142 to 147, wherein a line of sight between the at least one first ultrasound transducer and the at least one second ultrasound transducer is blocked by a refractive element.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A delivery assembly comprising:
a delivery apparatus comprising:
a balloon catheter defining a balloon catheter lumen;
an inflatable balloon mounted on the balloon catheter and defining a balloon cavity that is in fluid communication with the balloon catheter lumen, the balloon comprising:
a balloon proximal section attached to the balloon catheter;
a balloon intermediate section extending distally from the balloon proximal section; and
a balloon distal section extending distally from the balloon intermediate section; and
at least one ultrasound sensor configured to move between a first position which is axially offset from the balloon intermediate section, and a second position which is axially aligned with the balloon intermediate section inside the balloon cavity.

2. The delivery assembly of claim 1, wherein the balloon is configured to transition between a deflated state and an inflated state, wherein the ultrasound sensor is configured to be in the first position in the deflated state of the balloon, and to be in the second position in the inflated state of the balloon.

3. The delivery assembly of claim 1, wherein the delivery apparatus further comprises a spring configured to bias the at least one sensor to the second position.

4. The delivery assembly of claim 1, wherein the delivery apparatus further comprises a sleeve extending proximally from the at least one sensor.

5. The delivery assembly of claim 4, the at least one ultrasound sensor defines a sensor proximal end surface, and wherein, when inflation fluid is injected into the balloon catheter lumen, the inflation fluid is configured to impinge against the sensor proximal end surface, when in the first position, so as to distally bias the at least one ultrasound sensor towards the second position.

6. The delivery assembly of claim 1, wherein the delivery apparatus further comprises a nosecone; a nosecone shaft extending proximally from the nosecone through the balloon cavity and through the balloon catheter lumen; and a stopper disposed around the nosecone shaft inside the balloon cavity.

7. The delivery assembly of claim 6, wherein the at least one ultrasound sensor is configured to engage with the stopper when moved from the first to the second position, and is prevented from further axial movement in a direction from the first towards the second position by the stopper.

8. The delivery assembly of claim 1, wherein the at least one ultrasound sensor comprises at least two ultrasound transducers which are diametrically opposite to each other.

9. A delivery assembly comprising:
a delivery apparatus comprising:
a balloon catheter defining a balloon catheter lumen;
an inflatable balloon mounted on the balloon catheter and defining a balloon cavity that is in fluid communication with the balloon catheter lumen;
at least one ultrasound sensor; and
a temperature sensor.

10. The delivery assembly of claim 9, wherein the delivery apparatus further comprises a sensor data unit in communication with the at least one ultrasound sensor, wherein the sensor data unit is configured, based at least in part on an output of the at least one ultrasound sensor, to determine a diameter indication of the balloon.

11. The delivery assembly of claim 10, wherein the delivery apparatus further comprises a flow sensor, wherein the sensor data unit adjusts a temperature value output by the temperature sensor based at least in part on the output of the flow sensor, the determination of the diameter indication of the balloon based at least in part on the adjusted temperature value.

12. The delivery assembly of claim 10, wherein the sensor data unit is configured, based at least in part on an output of the at least one ultrasound sensor, to determine a diameter indication of an anatomical wall around the balloon.

13. The delivery assembly of claim 12, wherein the delivery apparatus further comprises a flow sensor, wherein the sensor data unit adjusts a temperature value output by the temperature sensor based at least in part on the output of the flow sensor, the determination of the diameter indication of the anatomical wall based at least in part on the adjusted temperature value.

14. The delivery assembly of claim 10, further comprising a prosthetic valve disposed around the balloon, wherein the sensor data unit is configured, responsive to an output of the at least one ultrasound sensor, to determine a diameter indication of the prosthetic valve.

15. A delivery assembly comprising:
a delivery apparatus comprising:
a balloon catheter defining a balloon catheter lumen;
an inflatable balloon mounted on the balloon catheter and defining a balloon cavity that is in fluid communication with the balloon catheter lumen;
at least one first ultrasound transducer; and
at least one second ultrasound transducer axially offset from the at least one first ultrasound transducer.

16. The delivery assembly of claim 15, wherein the delivery apparatus further comprises a sensor data unit in communication with the at least one first ultrasound transducer and the at least one second ultrasound transducer; and wherein the sensor data unit is configured to:
control the at least one first ultrasound transducer to generate ultrasound waves; and
detect the ultrasound waves at the at least one second ultrasound transducer.

17. The delivery assembly of claim 15, wherein the delivery apparatus further comprises a sensor data unit in communication with the at least one first ultrasound transducer and the at least one second ultrasound transducer; and wherein the sensor data unit is configured, based at least in part on the detected ultrasound waves, to determine a diameter indication of the balloon.

18. The delivery assembly of claim 15, wherein the delivery apparatus further comprises a sensor data unit in communication with the at least one first ultrasound transducer and the at least one second ultrasound transducer; and wherein the sensor data unit is configured, based at least in part on the detected ultrasound waves, to determine a diameter indication of an anatomical wall around the balloon.

19. The delivery assembly of claim 15, wherein the delivery apparatus further comprises a sensor data unit in communication with the at least one first ultrasound transducer and the at least one second ultrasound transducer; and wherein the delivery assembly further comprises a prosthetic valve disposed around the balloon, wherein the sensor data unit is configured, based at least in part on the detected ultrasound waves, to determine a diameter indication of the prosthetic valve.

20. The delivery assembly of claim 15, wherein a portion of the balloon comprises a refractive surface, the refractive surface of the balloon positioned between the at least one first ultrasound transducer and the at least one second ultrasound transducer.

21. The delivery assembly of claim 15, further comprising a refractive element comprising a refractive surface positioned between the at least one first ultrasound transducer and the at least one second ultrasound transducer such that a line of sight between the at least one first ultrasound transducer and the at least one second ultrasound transducer is blocked.
